(19) United States
(12) Patent Application Publication
(10) Pub. No.: US 2024/0352456 A1
(43) Pub. Date: Oct. 24, 2024

(54) COMPOSITIONS AND METHODS FOR TREATING SICKLE CELL DISEASES

(71) Applicant: THE UNITED STATES OF AMERICA, AS REPRESENTED BY THE SECRETARY, DEPARTMENT OF HEALTH & HUMAN SERVICES, Bethesda, MD (US)

(72) Inventors: John Fitzgerald TISDALE, Washington, DC (US); Bjorg GUDMUNDSDOTTIR, Silver Spring, MD (US); Laxminath TUMBURU, Rockville, MD (US)

(21) Appl. No.: 18/560,035

(22) PCT Filed: May 10, 2022
(86) PCT No.: PCT/US22/28510

§ 371 Date: Nov. 9, 2023

Related U.S. Application Data
(60) Provisional application No. 63/188,843, filed on May 14, 2021.
(60) Provisional application No. 63/188,320, filed on May 13, 2021.

(51) Int. Cl. C12N 15/113; A61K 31/713; A61K 35/12; A61P 7/06; C12N 15/86

(52) U.S. Cl. CPC C12N 15/113; A61K 31/713; A61K 35/12; A61P 7/06; C12N 15/86; A61K 2035/124; C12N 2310/531; C12N 2740/15011; C12N 2799/04

(57) ABSTRACT

The present invention provides ribonucleotide agents that decrease expression of RIOK3 for the treatment of a sickle cell disease.

### CROSS-REFERENCE TO RELATED APPLICATIONS

This international application claims the benefit of U.S. Provisional Patent Application No. 63/188,320, filed on May 13, 2021, and U.S. Provisional Patent Application No. 63/188,843 filed on May 14, 2021, which are incorporated by reference herein in their entireties.

### REFERENCE TO SEQUENCE LISTING SUBMITTED ELECTRONICALLY

Pursuant to the EFS-Web legal framework and 37 CFR §§ 1.821-825 (see MPEP § 2442.03 (a)), a Sequence Listing in the form of an ASCII-compliant text file (entitled “3000093-006977_Sequence_Listing_ST25” created on May 4, 2022 and 6,000 bytes in size) is submitted concurrently with the instant application, and the entire contents of the Sequence Listing are incorporated herein by reference.

### BACKGROUND

### 1. Field

The present disclosure relates to ribonucleotide agents that decrease the expression of serine/threonine-protein kinase RIO3 encoded by the RIOK3 gene. The ribonucleotide agents specifically target the RIOK3 mRNA, preventing it from being translated, lowering the expression of serine/threonine-protein kinase RIO3, and subsequently, its biological function, and can be used as a therapeutic agent in patients with a sickle cell disease and complications thereof.

### 2. Description of Related Art

Hemoglobin is an oxygen-transport metalloprotein in erythrocytes comprising four protein subunits comprising two alpha-globin subunits and two beta-globin subunits. Alpha-hemoglobin is encoded by the HBA1 and HBA2 genes. Beta-hemoglobin is encoded by the HBB gene. In humans, epsilon-globin is expressed during the embryonic stage and gamma-globin is expressed during the fetal stage. After birth, gamma-globin expression decreases and beta-globin increases. Hardison Cold Spring Harb Perspect Med (2012) 2: a011627.

Fetal red blood cells (RBCs), which contain HbF (α₂γ₂), have higher affinity for oxygen than adult RBCs, which contain hemoglobin A (HbA; α₂β₂), and this facilitates transfer of oxygen from the maternal to the fetal circulation. The switch from production of γ to β globin begins in utero and results in the linear decline of HbF in the fetal RBC population, such that HbF levels of 50-95% at birth decline to <5% by six months of life. Colombo et al. Br J Haematol. (1976) 32:79-87.

Sickle cell disease (SCD) is a group of inherited red blood cell disorders characterized by erythrocytes that form a “sickle” shape when deoxygenated, have a shortened lifespan, and thereby cause a constant shortage of erythrocytes. The clinical complications of SCD include acute and chronic pain, infection, acute chest syndrome, stroke, multiorgan failure, and premature death. Centers for Disease Control and Prevention “Sickle Cell Disease (SCD)” (2021).

In patients with sickle cell disease, at least one of the beta-globin subunits in hemoglobin is replaced with hemoglobin S, either inherited in a homozygous manner (HbSS, sickle cell anemia), or inherited with another abnormal beta-globin subunit. HbSS is the most severe form of SCD, followed by HbS-beta zero thalassemia, though complications continue to occur in other forms of the disease such as HbSC or HbS-beta plus thalassemia, among others. MedlinePlus “Sickle Cell Disease” (2021). There exists a need in the art for treating beta-globinopathies.

### BRIEF SUMMARY OF THE INVENTION

The invention provides for a ribonucleotide agent that decreases expression of RIOK3. The ribonucleotide agent may be an antisense oligonucleotide, short hairpin RNA (shRNA), small interfering RNA (siRNA), optionally an asymmetrical iRNA (aiRNA), a microRNA, a miniRNA, a lncRNA, ribozyme, or a combination thereof.

In an embodiment, the ribonucleotide agent may be a short hairpin RNA (shRNA). The shRNA may comprise a forward sequence of SEQ ID NO: 3 and/or a reverse sequence of SEQ ID NO: 4.

In an embodiment, the ribonucleotide agent may further decrease the expression of BLC11A. The ribonucleotide agent may further decrease the expression of LRF. The ribonucleotide agent may further increase the expression of POGZ.

In an embodiment, the ribonucleotide agent may further increase the expression of hemoglobin gamma, optionally HBG1, HGB2, or both.

In an embodiment, the ribonucleotide agent may further decrease the expression of hemoglobin beta (HBB).

In an embodiment, the ribonucleotide agent may target the mRNA sequence of SEQ ID NO: 1. The ribonucleotide agent may target the mRNA sequence of SEQ ID NO: 2. The target may further comprise flanking sequences 1-20 ribonucleotides 3′ and/or 5′ of SEQ ID NO: 2.

In an embodiment, a composition may comprise a ribonucleotide agent described herein. The composition may be a pharmaceutical composition. The composition may further comprise an adjuvant, carrier, buffers, antioxidants, wetting agents, lubricating agents, gelling agents, thickening agents, binding agents, disintegrating agents, humectants, preservatives, diluent, stabilizer, filler, excipient, or a combination thereof.

In an embodiment, a microparticle may comprise a ribonucleotide agent described herein. The microparticle may be a microsphere, microcapsule, nanosphere, nanocapsule, or a nanoparticle. The microparticle may comprise a lipid carrier.

In an embodiment, a composition may comprise a microparticle comprising a ribonucleotide agent described herein.

In an embodiment, a vector may comprise a polynucleotide encoding a ribonucleotide agent described herein. The vector may be an expression vector. The vector may be a viral vector. The viral vector may be a retroviral vector, adenoviral vector, adeno-associated viral vector, or lentiviral vector. The viral vector may be a lentiviral vector.

In an embodiment, a lentiviral vector may comprise a polynucleotide encoding a ribonucleotide agent described herein.

In an embodiment, the vector may further comprise a promoter. The vector may further comprise an erythroid specific promoter. The erythroid specific promoter may be alpha-spectrin promoter, ankyrin-1 promoter, gamma-globin promoter, or beta-globin promoter. The promoter may be a type III RNA polymerase III promoter. The promoter may be a U6 or H1 promoter. The promoter may be a tRNA or CMV promoter.

In an embodiment, the vector may comprise an enhancer, for example, an erythroid specific enhancer.

In an embodiment, a host cell may comprise a vector that comprises nucleic acid sequence that encodes a ribonucleotide agent described herein.

In an embodiment, a method for treating a sickle cell disease in a patient may comprise administration of an effective amount of a ribonucleotide agent described herein. The sickle cell disease may be hemoglobin SS disease, hemoglobin SC disease, hemoglobin SB+ (beta) thalassemia, hemoglobin SB 0 (beta-zero) thalassemia, hemoglobin SD, hemoglobin SE, or hemoglobin SO. The sickle cell disease may be Sickle Cell Anemia (SS), Sickle Hemoglobin-C Disease (SC), Sickle Beta-Plus Thalassemia or Sickle Beta-Zero Thalassemia.

In an embodiment, a method for treating a complication of sickle cell disease in a patient may comprise administration of an effective amount of a ribonucleotide agent described herein. The complication of sickle cell disease may be sickle cell crisis, vaso-occlusive crisis, acute chest syndrome, aplastic crisis, hemolytic crisis, dactylitis, acute chest syndrome, seizure, stroke, ischemia, transient ischemic attack, ischemic colitis, or a combination thereof.

In an embodiment, a method for promoting beta-globin synthesis in a cell may comprise administration of an effective amount of a ribonucleotide agent described herein.

In an embodiment, a method for treating a sickle cell disease in a patient comprising administration of an effective amount of a ribonucleotide agent described herein, a composition described herein, a microparticle described herein, a vector described herein, or a combination thereof.

In an embodiment, a method for treating a complication of sickle cell disease in a patient comprising administration of an effective amount of a ribonucleotide agent described herein, a composition described herein, a microparticle described herein, a vector described herein, or a combination thereof.

In an embodiment, a method for promoting beta-globin synthesis in a cell comprising administration of an effective amount of a ribonucleotide agent described herein, a composition described herein, a microparticle described herein, a vector described herein, or a combination thereof.

In an embodiment, an ex vivo method for treating a sickle cell disease in a patient in need thereof comprising (a) obtaining hematopoietic stem and progenitor cells, optionally a hemocytoblast (a hematopoietic stem cell), from a patient with a sickle cell disease; (b) administration of an effective amount of a ribonucleotide agent described herein, a composition described herein, a microparticle described herein, a vector described herein, or a combination thereof to the hematopoietic stem and progenitor cells to transfect the cells with the ribonucleotide agent; and (c) returning the transfected hematopoietic stem and progenitor cells to the patient. The administration of an effective amount of the ribonucleotide agent described herein, the composition described herein, the microparticle described herein, the vector described herein, or a combination thereof to the hematopoietic stem and progenitor cells is for a sufficient time to allow transfection of the hematopoietic stem and progenitor cells. The hematopoietic stem and progenitor cells may be CD34+. The hematopoietic stem and progenitor cells may be hemocytoblasts (hematopoietic stem cells).

In an embodiment, an isolated nucleotide may comprise the nucleic acid sequence of SEQ ID NO: 3.

In an embodiment, an isolated nucleotide may comprise the nucleic acid sequence of SEQ ID NO: 4.

In an embodiment, an isolated nucleotide may comprise the nucleic acid sequence of SEQ ID NO: 5.

In an embodiment, an isolated lentiviral vector may comprise a shRNA comprising a forward sequence comprising the nucleic acid sequence of SEQ ID NO: 3 and a reverse sequence comprising the nucleic acid sequence of SEQ ID NO: 4.

In one embodiment, an isolated nucleic acid may comprise the ribonucleotide sequence of SEQ ID NO: 1. A vector may comprise the nucleic acid encoding the ribonucleotide sequence of SEQ ID NO: 1. The vector may be expression vector. The vector may be a viral vector, optionally a retroviral vector, adenoviral vector, adeno-associated viral vector, or lentiviral vector.

In one embodiment, an isolated nucleic acid may comprise the ribonucleotide sequence of SEQ ID NO: 2. A vector may comprise the nucleic acid encoding the ribonucleotide sequence of SEQ ID NO: 2. The vector may be expression vector. The vector may be a viral vector, optionally a retroviral vector, adenoviral vector, adeno-associated viral vector, or lentiviral vector.

### BRIEF DESCRIPTION OF THE DRAWINGS

FIG. 1 depicts human RIOK3 domain structure according to SMART analysis.

FIG. 2A-C depicts that RIOK3 is expressed in early adult erythroid cells according to the BioGPS database.

FIG. 3 depicts that RIOK3 expression is confined to developing erythroid cells in the Bloodspot database (3 out of 4 probes).

FIG. 4 depicts that RIOK3 is expected to have multiple transcript variants which could be due to regulation at the transcriptional, translational, or post-translational level. To increase fetal hemoglobin expression, it is preferred that knockdown of RIOK3 be directed to specific regions at the 3′-UTR. For example, the inventors found that targeting exons 6 or 10 or the 3′-UTR at different location does not result in fetal globin upregulation.

FIG. 5 depicts that RIOK3 knockdown results in marked upregulation of hemoglobin fetal (gamma-subunit), HBG1 and HBG2 expression. Hemoglobin beta (HBB) expression is decreased. CD34+ hematopoietic stem and progenitor cell derived erythroblasts were transduced on day 2 of culture with a control lentiviral vector (ShNC) or a RIOK3 specific lentiviral vector (ShR3) targeting the 3′-UTR and hemoglobin beta (HBB), hemoglobin alpha (HBA), hemoglobin gamma-1 (HBG1), and hemoglobin gamma-2 (HBG2) levels analyzed on day 17 of culture by high performance liquid chromatography (HPLC) (upper panels). % HbF was calculated by dividing HBG1+HBG2 values with total β-globin (HBB+HBG1+HBG2) values (bottom panel). The data shows that upon RIOK3 knockdown the levels of fetal-globins HBG1 and HBG2 are robustly increased compared to control.

FIG. 6 depicts decreased RIOK3, BCL11A, and LRF RNA expression after shRNA knockdown. CD34+ hematopoietic stem and progenitor cell derived erythroblasts were transduced on day 2 of culture with a control shRNA lentiviral vector (ShNC) or a RIOK3 specific lentiviral vector (ShR3) targeting the 3′-UTR and BCLI IA and LRF expression analyzed on day 12 of culture by Q-PCR. RIOK3 encodes serine/threonine-protein kinase RIO3. BCL11A encodes B-cell lymphoma/leukemia 11A. LRF encodes lymphoma/leukemia-related factor.

FIG. 7 depicts significant reduction in RIOK3, BCL11A, and LRF protein levels upon RIOK3 knockdown in primary human erythroid progenitor cells. CD34+ hematopoietic stem and progenitor cell derived erythroblasts were transduced on day 2 of culture with a control shRNA lentiviral vector (ShNC) or a RIOK3 specific lentiviral vector (ShR3) targeting the 3′-UTR and BCLI IA and LRF protein levels analyzed on day 12 of culture by western blotting.

FIG. 8 depicts that Cytospin analysis shows no morphological differences on day 15 of culture between cells transduced with control shRNA (ShNC) vs cells transduced with RIOK3 specific shRNA (ShR3) targeting the 3′-UTR.

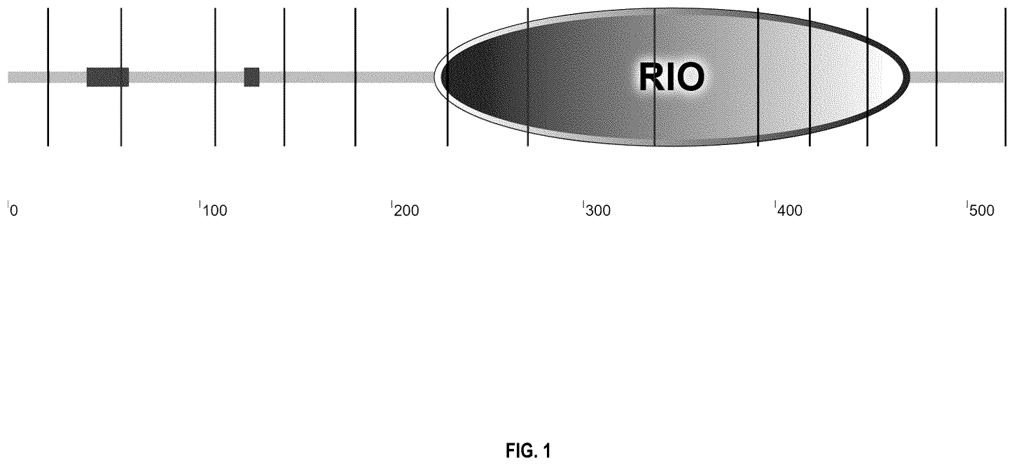

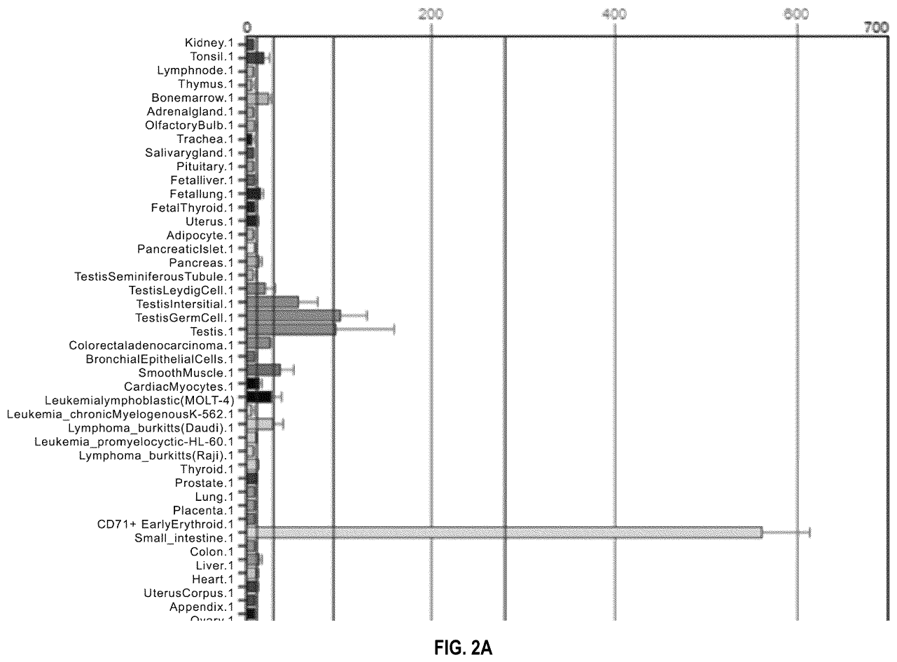

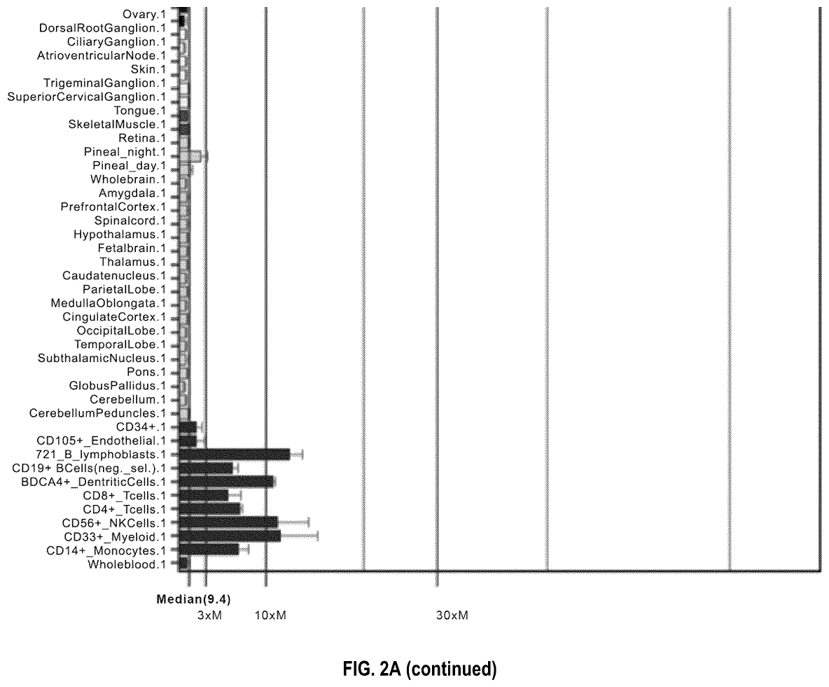

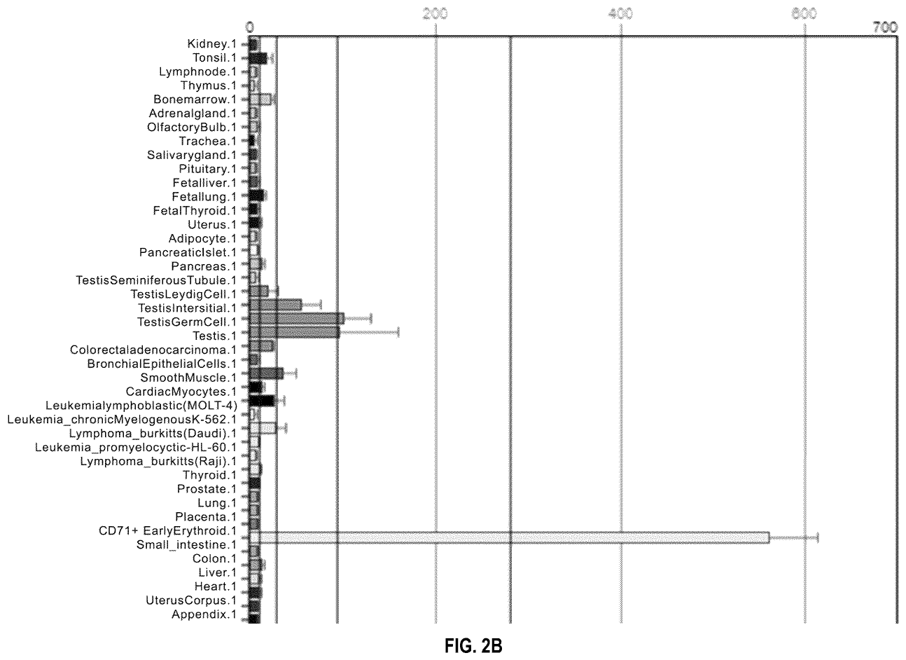

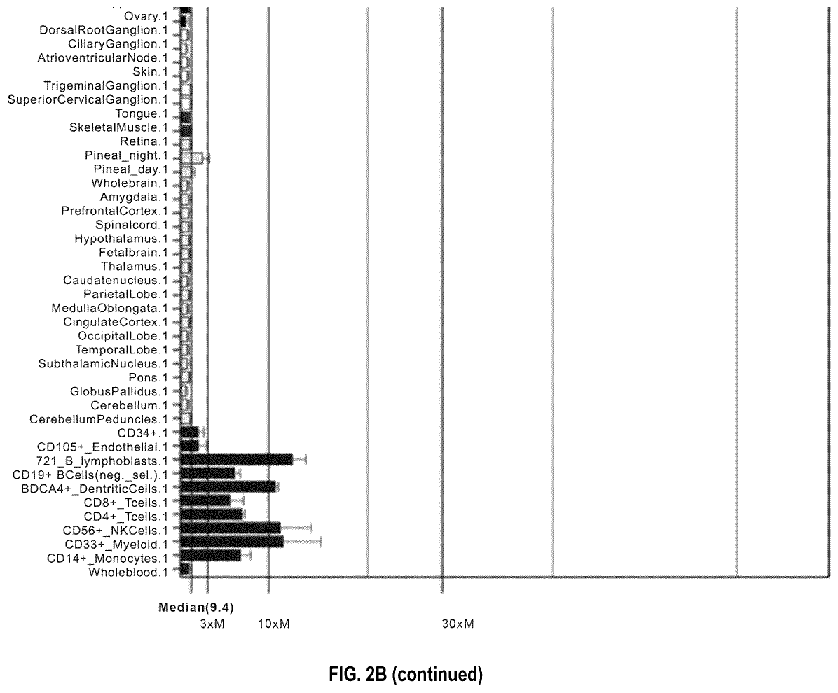

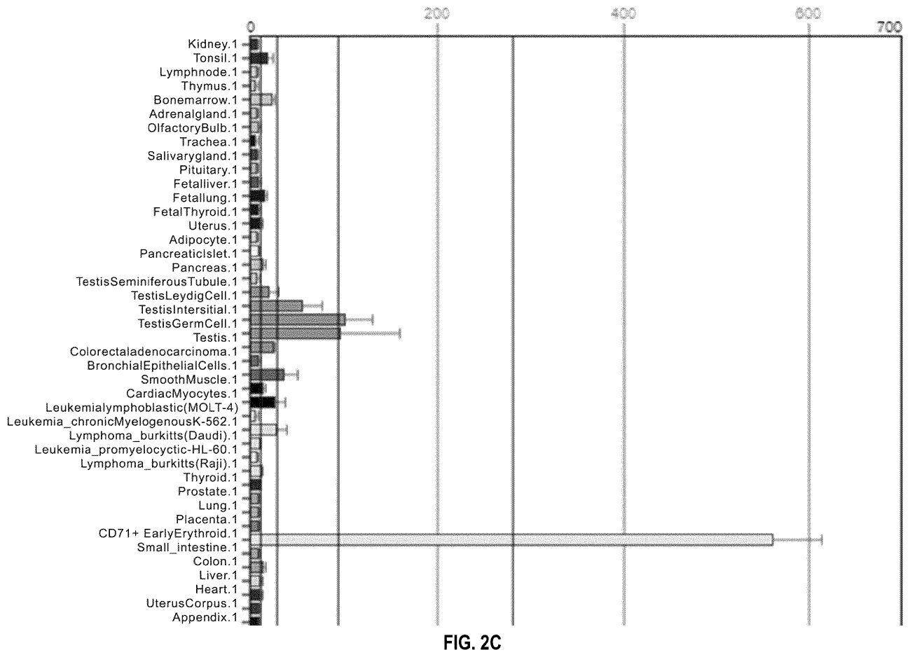

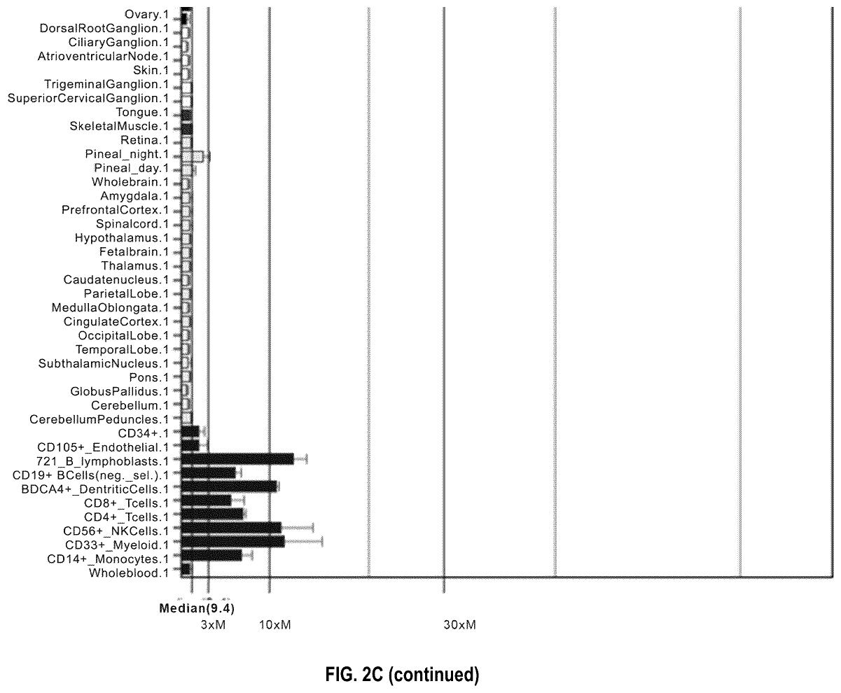

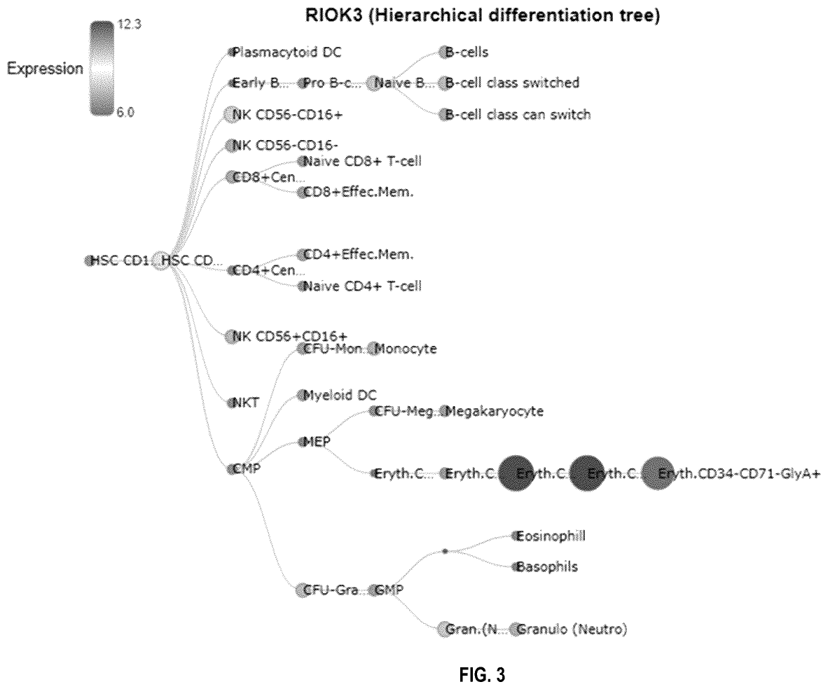

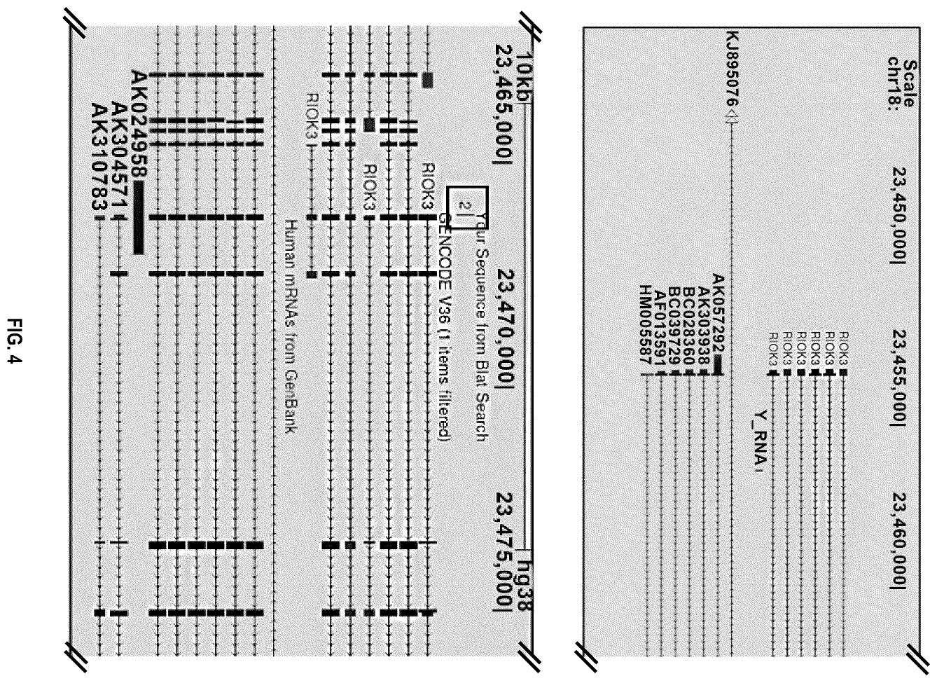

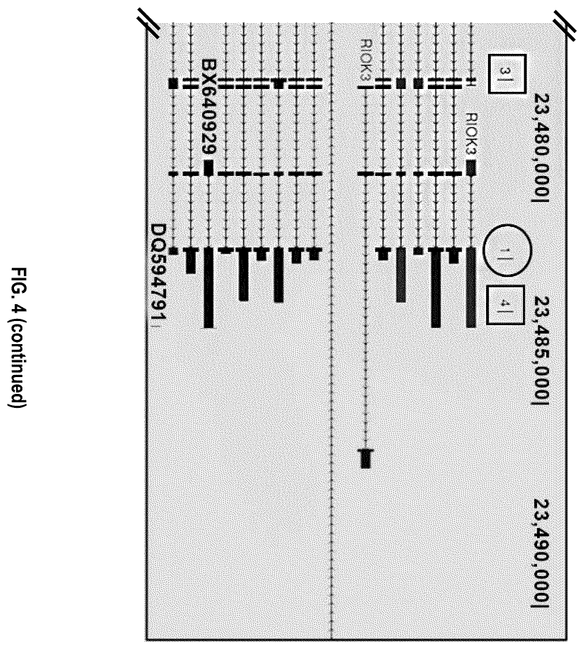

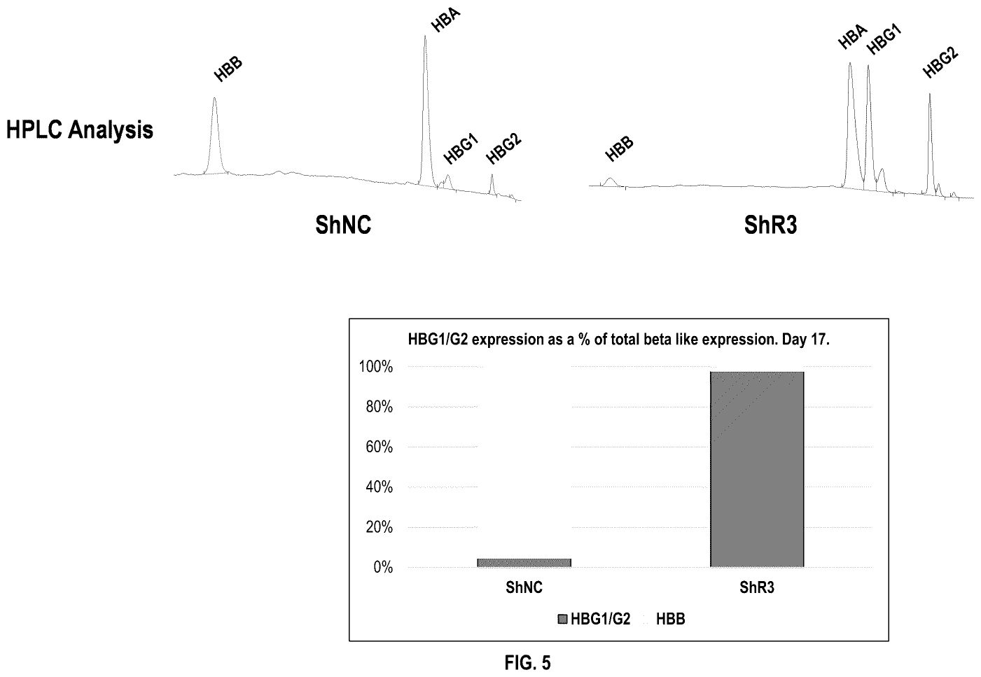

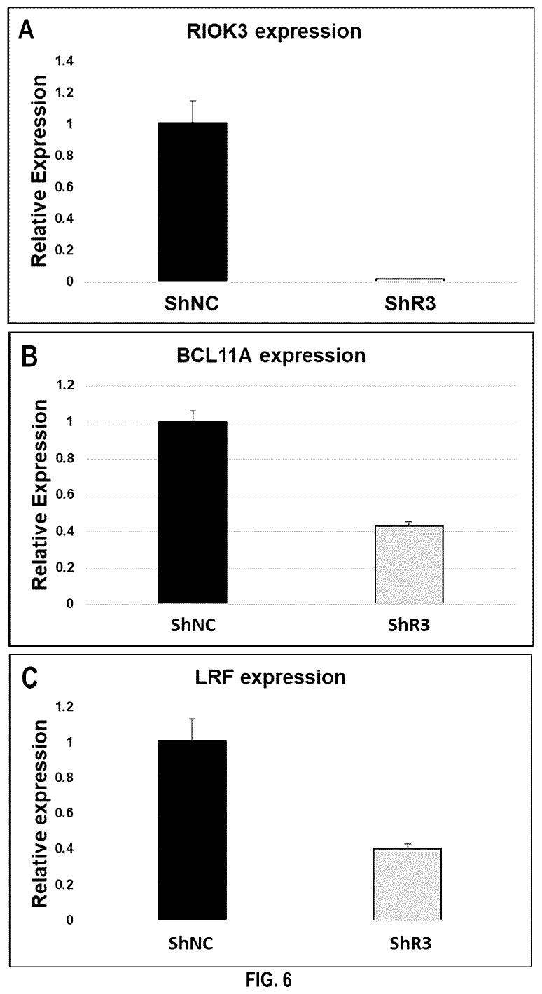

### DETAILED DESCRIPTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments, and is not intended to be limiting. Instead, the scope of the present disclosure will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

“Effective amount,” as used herein, refers broadly to the amount of a compound, antibody, antigen, or cells that, when administered to a patient for treating a disease, is sufficient to effect such treatment for the disease. The effective amount may be an amount effective for prophylaxis, and/or an amount effective for prevention. The effective amount may be an amount effective to reduce, an amount effective to prevent the incidence of signs/symptoms, to reduce the severity of the incidence of signs/symptoms, to eliminate the incidence of signs/symptoms, to slow the development of the incidence of signs/symptoms, to prevent the development of the incidence of signs/symptoms, and/or effect prophylaxis of the incidence of signs/symptoms. The “effective amount” may vary depending on the disease and its severity and the age, weight, medical history, susceptibility, and pre-existing conditions, of the patient to be treated. The term “effective amount” is synonymous with “therapeutically effective amount” for purposes of this invention.

“Host cell,” as used herein, refers broadly to the particular subject cell transfected with a nucleic acid molecule and the progeny or potential progeny of such a cell. Progeny may not be identical to the parent cell transfected with the nucleic acid molecule due to mutations or environmental influences that may occur in succeeding generations or integration of the nucleic acid molecule into the host cell genome.

“Mammal,” as used herein, refers broadly to any and all warm-blooded vertebrate animals of the class Mammalia, characterized by a covering of hair on the skin and, in the female, milk-producing mammary glands for nourishing the young. Mammals include, but are not limited to, humans, domestic and farm animals, and zoo, sports, or pet animals. Examples of mammals include but are not limited to alpacas, armadillos, capybaras, cats, camels, chimpanzees, chinchillas, cattle, dogs, gerbils, goats, gorillas, guinea pigs, hamsters, horses, humans, lemurs, llamas, mice, non-human primates, pigs, rats, sheep, shrews, squirrels, and tapirs. Mammals include but are not limited to bovine, canine, equine, feline, murine, ovine, porcine, primate, and rodent species. Mammal also includes any and all those listed on the Mammal Species of the World maintained by the National Museum of Natural History, Smithsonian Institution in Washington D.C. Similarly, the term “subject” or “patient” includes both human and veterinary subjects and/or patients.

“Short interfering RNA” (siRNA), also referred to herein as “small interfering RNA” is an agent which functions to inhibit expression of a target gene. These are the effector molecules for inducing RNAi, leading to posttranscriptional gene silencing with RNA-induced silencing complex (RISC). In addition to siRNA, which can be chemically synthesized, various other systems in the form of potential effector molecules for posttranscriptional gene silencing are available, including short hairpin RNAs (shRNAs), long dsRNAs, short temporal RNAs, and micro RNAs (miRNAs). These effector molecules either are processed into siRNA, such as in the case of shRNA, or directly aid gene silencing, as in the case of miRNA. The present invention thus encompasses the use of shRNA as well as any other suitable form of RNA to effect posttranscriptional gene silencing by RNAi. Use of shRNA has the advantage over use of chemically synthesized siRNA in that the suppression of the target gene is typically long-term and stable. An siRNA may be chemically synthesized, may be produced by in vitro by transcription, or may be produced within a host cell from expressed shRNA.

“Gene silencing,” as used herein, induced by the ribonucleotide agent that refers broadly to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without introduction of RNA interference. In one embodiment, the mRNA levels are decreased by at least about 70%, about 80%, about 90%, about 95%, about 99%, about 100% by the ribonucleotide agent described herein.

“Inhibition of target gene expression” or “inhibition of marker gene expression,” as used herein, refers broadly to any decrease in expression or protein activity or level of the target gene or protein encoded by the target gene. The decrease may be of at least 30%, 40%, 50%, 60%, 70%, 80%, 90%, 95% or 99% or more as compared to the expression of a target gene or the activity or level of the protein encoded by a target gene which has not been targeted by an RNA interfering agent.

“Substantially free,” as used herein, refers broadly to the presence of a specific component in an amount less than 1%, preferably less than 0.1% or 0.01%. More preferably, the term “substantially free” refers broadly to the presence of a specific component in an amount less than 0.001%. The amount may be expressed as w/w or w/v depending on the composition.

“Treatment,” as used herein, refers broadly to both therapeutic treatment and prophylactic or preventative measures. Those in need of treatment include those already with the disorder as well as those in which the disorder is to be prevented. As used herein, the term “treating,” refers broadly to treating a disease, arresting, or reducing the development of the disease or its clinical symptoms, and/or relieving the disease, causing regression of the disease or its clinical symptoms. Therapy encompasses prophylaxis, treatment, remedy, reduction, alleviation, and/or providing relief from a disease, signs, and/or symptoms of a disease. Therapy encompasses an alleviation of signs and/or symptoms in patients with ongoing disease signs and/or symptoms. Therapy also encompasses “prophylaxis”. The term “reduced”, for purpose of therapy, refers broadly to the clinical significant reduction in signs and/or symptoms. Therapy includes treating relapses or recurrent signs and/or symptoms. Therapy encompasses but is not limited to precluding the appearance of signs and/or symptoms anytime as well as reducing existing signs and/or symptoms and eliminating existing signs and/or symptoms. Therapy includes treating chronic disease (“maintenance”) and acute disease. For example, treatment includes treating or preventing relapses or the recurrence of signs and/or symptoms.

### Treatment of Sickle Cell Diseases by Downregulation of RIOK3

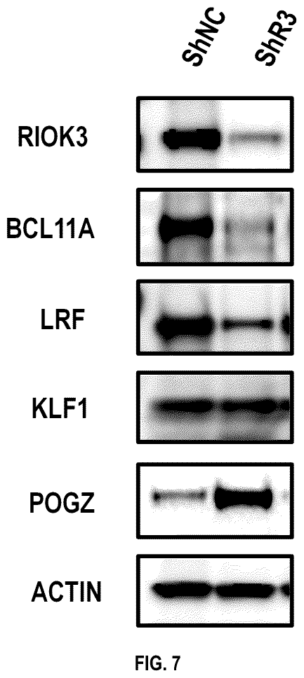

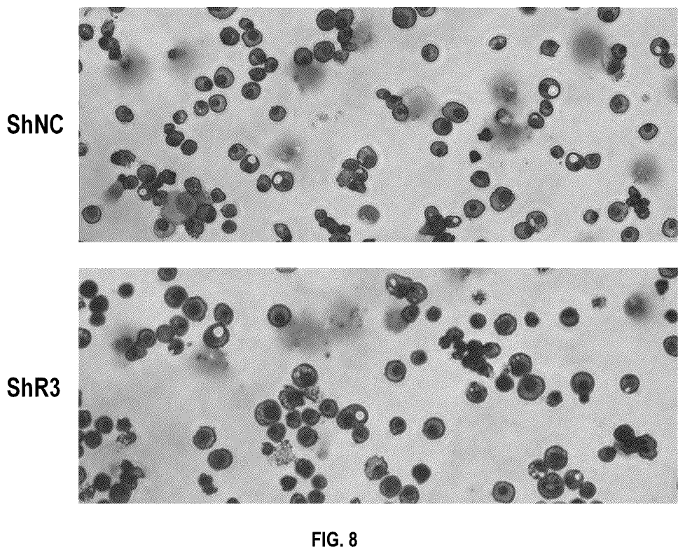

RIO kinase 3, a serine/threonine kinase and a member of the right open reading frame (RIO) kinase family, plays a role as a downstream effector of transcription factor Pogo transposable element with Zinc finger domain (POGZ) in the negative regulation of beta-globin (HBB) synthesis. The inventors surprisingly discovered that the downregulation of the expression of RIOK3 leads to a subsequent down regulation of the expression of BCL11A and LRF, and an upregulation in the expression of POGZ. See, e.g., FIG. 7. The downregulation of RIOK3 expression by administration of a ribonucleotide agent also leads to the upregulation of expression of hemoglobin gamma-1 (HBG1) and hemoglobin gamma-2 (HBG2) and a downregulation of the expression of hemoglobin beta (HBB). The upregulation of HBG1 and HBG2 may reduce the severity of sickle cell disease. Elevated fetal hemoglobin, specifically hereditary persistence of fetal hemoglobin (HPFH), in the clinical course of patients with sickle cell disease, has shown protective effects. Those patients who inherit a mutation, for example, in fetal hemoglobin, leading to persistent elevation in HbF (HPFH) are protected from the complications of sickle cell disease. It was also unexpected that the administration of the ribonucleotide agents described herein also reduce the incidence and/or severity of sickle cell disease complications and susceptibility to malaria.

TABLE 1
Sequences

| Sequence | SEQ ID NO |
|---|---|
| Homo sapiens RIO kinase 3 (RIOK3), | 1 |
| transcript variant 1, mRNA | |
| Homo sapiens RIO kinase 3 (RIOK3), | 2 |
| transcript variant 1, mRNA (3′-UTR | (GCTCAGCATTG |
| Sequence) | AGAGAATAAA) |

### Ribonucleotide Agent

The ribonucleotide agent that decreases RIOK3 described herein may be an antisense oligonucleotide, shRNA, small interfering RNA (siRNA), optionally an asymmetrical iRNA (aiRNA), a microRNA, a miniRNA, a lncRNA, ribozyme, or a combination thereof.

The ribonucleotide agents described herein may be delivered by any suitable means including but not limited to viral vectors, micelles, lipid delivery, polymer compositions, or a combination thereof. Song & Yang N Am J Med Sci (2010) 2 (12): 598-601. The ribonucleotide agent may be encoded by a polynucleotide in a vector, optionally a viral vector. For example, a viral vector, optionally a lentiviral vector, may comprise a polynucleotide that encodes the ribonucleotide agent described herein, preferably driven by an erythroid specific promoter (e.g., alpha-spectrin promoter, ankyrin-1 promoter, gamma-globin promoter, the beta-globin promoter), optionally in combination with one or more enhancers, for example, one or more erythroid specific enhancers.

The inventor surprisingly discovered that to increase fetal hemoglobin expression, it is preferred that knockdown of RIOK3 be directed to specific regions at the 3′-UTR (SEQ ID NO: 2). For example, the inventors found that targeting exons 6 or 10 or the 3′-UTR at different location does not result in fetal globin upregulation.

The ribonucleotide agent described herein may, for example, target the 3′UTR sequence of the RIOK3 mRNA, preferably the ribonucleotide sequence of SEQ ID NO: 2, or the ribonucleotide sequence of SEQ ID NO: 2 including 1-20 ribonucleotides 3′ or 5′ of the ribonucleotide sequence of SEQ ID NO: 2. The ribonucleotide agent described herein may target a ribonucleotide sequence comprising 1-20 ribonucleotides 3′ of the sequence of SEQ ID NO: 2 and the ribonucleotide sequence of SEQ ID NO: 2. The ribonucleotide agent described herein may target a ribonucleotide sequence comprising 1-20 ribonucleotides 5′ of the sequence of SEQ ID NO: 2 and the ribonucleotide sequence of SEQ ID NO: 2. These regions 5′ and 3′ may be described as “flanking sequences” around the sequence of SEQ ID NO: 2. For example, the flanking sequence may comprise 1, 2, 3, 4, 5, 6, 7, 8, 9, 10, 11, 12, 13, 14, 15, 16, 17, 18, 19, or 20 nucleotides 5′ and/or 3′ of SEQ ID NO: 2 (GCTCAGCATTGAGAGAATAAA).

The ribonucleotide agent described herein may bind to, and, optionally, cleave the target sequence RIOK3, optionally SEQ ID NO: 1, preferably SEQ ID NO: 2 (GCTCAGCATTGAGAGAATAAA).

### RNAi

The RNA interference (RNAi) pathway is used by cells to regulate the activity of many genes. RNAi, also called post transcriptional gene silencing (PTGS), refers to the biological process in which RNA molecules inhibit gene expression. An “RNA interfering agent” as used herein is any agent that interferes with or inhibits expression of a target gene by RNA interference (RNAi). Such RNA interfering agents include, but are not limited to, nucleic acid molecules including RNA molecules which are homologous to a target gene or a fragment thereof, short interfering RNA (siRNA), short hairpin RNA (shRNA), and small molecules which interfere with or inhibit expression of a target gene by RNA interference (RNAi).

RNAi is a process by which the expression or introduction of RNA of a sequence that is identical or highly similar to a target gene results in the sequence-specific degradation or PTGS of messenger RNA (mRNA) transcribed from that targeted gene, thereby inhibiting expression of the target gene. This process has been described in plants, invertebrates, and mammalian cells. RNAi can also be initiated by introducing nucleic acid molecules, e.g., synthetic siRNAs or RNA interfering agents, to inhibit or silence the expression of target genes.

The RNAi agents described herein may bind to, and, optionally, cleave the target sequence RIOK3, optionally SEQ ID NO: 1, preferably SEQ ID NO: 2 (GCTCAGCATTGAGAGAATAAA).

shRNA

The ribonucleotide agent may be a siRNA is a small hairpin (also called stem loop) RNA (shRNA). These shRNAs are composed of a short (e.g., 19-25 nucleotides) antisense strand, followed by a 5-9 nucleotide loop, and the complementary sense strand. Alternatively, the sense strand may precede the nucleotide loop structure and the antisense strand may follow. These shRNAs may be contained in plasmids, retroviruses, and lentiviruses. For example, the shRNA may be delivered to a patient via a lentivirus vector driven by an erythroid specific promoter in conjunction with an erythroid specific enhancer.

The shRNA may comprise:

Forward sequence (SEQ ID NO: 3):
5′-CCGGGCTCAGCATTGAGAGAATAAACTCGAGTTTATTCTCTC
AATGCTGAGCTTTTTG-3′
Reverse sequence (SEQ ID NO: 4):
5′-AATTCAAAAAGCTCAGCATTGAGAGAATAAACTCGAGTTTAT
TCTCTCAATGCTGAGC-3′

A shRNA may comprise the forward sequence of SEQ ID NO: 3 and the reverse sequence of SEQ ID NO: 4, using a replication incompetent lentiviral system (e.g., MISSION® pLKO.1 system) that comprises an U6 promoter to drive the transcription of the shRNA. The U6 promoter is preferred for transcribing shRNAs because it is PolIII dependent, the RNA is not polyadenylated and therefore the hairpin is more efficiently generated.

A shRNA construct may comprise a shRNA comprising the forward sequence of SEQ ID NO: 3 and the reverse sequence of SEQ ID NO: 4 in a lentiviral system driven by an erythroid specific promoter may be used in methods for decreasing the expression of RIOK3. The shRNA construct may be used to transfect autologous CD34+ hematopoietic stem and progenitor cells (HSPCs) isolated from a patient suffering from a sickle cell disease. The shRNA construct decreases the expression of RIOK3 and, consequently, the expression of hemoglobin-beta, while increasing the expression of hemoglobin-gamma. The transfected HSPCs are returned to the patient thereby treating the sickle cell disease, and/or complications thereof.

### Antisense Oligonucleotides

The ribonucleotide agent may be an antisense oligonucleotide. Antisense oligonucleotides are relatively short nucleic acids that are complementary (or antisense) to the coding strand (sense strand) of the mRNA encoding RIOK3, optionally SEQ ID NO: 1. Preferably, the ribonucleotide agent targets a subsection of the mRNA encoding RIOK3, preferably SEQ ID NO: 2. Antisense oligonucleotides may be RNA based, DNA based, or a RNA/DNA hybrid. Also, antisense oligonucleotides may be modified to increase their stability.

Without being bound by theory, the binding of these relatively short oligonucleotides to the mRNA is believed to induce stretches of double stranded RNA that trigger degradation of the messages by endogenous RNAses. Additionally, sometimes the oligonucleotides are specifically designed to bind near the promoter of the coding sequence, and under these circumstances, the antisense oligonucleotides may additionally interfere with translation of the mRNA. Regardless of the specific mechanism by which antisense oligonucleotides function, their administration to a cell or tissue allows the degradation of the mRNA encoding RIOK3. Accordingly, antisense oligonucleotides decrease the expression and/or activity of RIOK3. This decrease in the expression/activity of RIOK3, leads to decrease in hemoglobin beta (HBB) expression and an increase in hemoglobin gamma (HBG1, HBG2) expression. Specifically, it is preferred that the ribonucleotide agents be directed to specific regions at the 3′-UTR (SEQ ID NO: 2) because targeting exons 6 or 10 or the 3′-UTR at different location does not result in fetal globin upregulation.

The ribonucleotide agents described herein may further decrease the expression of BCL11A. The ribonucleotide agent may further decrease the expression of LRF. The ribonucleotide agent may further increase the expression of POGZ.

In an embodiment, the ribonucleotide agent may further increase the expression of hemoglobin gamma, optionally HBG1, HGB2, or both.

The antisense oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, and serum half-life. The oligonucleotide may include other appended groups including but not limited to peptides (e.g., for targeting host cell receptors), or agents facilitating transport across the cell membrane (See, e.g., Letsinger et al. (1989) Proc. Natl. Acad. Sci. U.S.A. 86:6553-6556; Lemaitre et al. (1987) Proc. Natl. Acad. Sci. 84:648-652; WO 88/09810) or the blood-brain barrier (see, e.g., WO 89/10134), hybridization-triggered cleavage agents (See, e.g., Krol et al., 1988, BioTechniques 6:958-976) or intercalating agents (see, e.g., Zon, 1988, Pharm. Res. 5:539-549). To this end, the oligonucleotide can be conjugated to another molecule.

Antisense oligonucleotides described herein may be synthesized by standard methods known in the art, e.g., by use of an automated DNA synthesizer (such as are commercially available from Biosearch, Applied Biosystems). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports. Sarin et al. (1988) Proc. Natl. Acad. Sci. U.S.A. 85:7448-7451.

A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

Another approach utilizes a recombinant DNA construct in which the antisense oligonucleotide is placed under the control of a strong pol III or pol II promoter to achieve intracellular concentrations of the antisense sufficient to suppress translation on endogenous mRNAs. For example, a vector can be introduced in vivo such that it is taken up by a cell and directs the transcription of an antisense RNA. The vectors can remain episomal or become chromosomally integrated, and still be transcribed to produce the desired antisense RNA. Suitable vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Suitable promoters can be inducible or constitutive. Suitable promoters include but are not limited to: the SV40 early promoter region (Bernoist and Chambon (1981) Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), or a combination thereof. Any type of plasmid, cosmid, YAC or viral vector can be used to prepare the recombinant DNA construct that can be introduced directly into the tissue site. Viral vectors can be used which selectively infect the desired tissue, in which case administration may be accomplished by another route (e.g., systematically). The antisense oligonucleotides described herein may decrease RIOK3 by targeting SEQ ID NO: 1, preferably SEQ ID NO: 2.

The antisense oligonucleotide may comprise the antisense strand to the sense strand of the ribonucleotide sequence of SEQ ID NO: 1, preferably SEQ ID NO: 2. A composition, including a pharmaceutical composition may comprise an antisense oligonucleotide that comprises the antisense strand to the sense strand of the ribonucleotide sequence of SEQ ID NO: 1, preferably SEQ ID NO: 2. A microparticle, including a lipid comprising microparticle, may comprise an antisense oligonucleotide that comprises the antisense strand to the sense strand of the ribonucleotide sequence of SEQ ID NO: 1, preferably SEQ ID NO: 2. For example, the antisense oligonucleotide may bind to SEQ ID NO: 2 (GCTCAGCATTGAGAGAATAAA), leading to the mRNA's degradation.

### Small Interfering RNA

The ribonucleotide agent may be a small interfering RNA (siRNA or RNAi) molecule. RNAi constructs comprise double stranded RNA that can specifically block expression of a target gene. “RNA interference” or “RNAi” is a term initially applied to a phenomenon where double-stranded RNA (dsRNA) blocks gene expression in a specific and post-transcriptional manner. RNAi provides a useful method of inhibiting gene expression in vitro or in vivo. RNAi constructs include but are not limited to small interfering RNAs (siRNAs), asymmetrical interfering RNA (aiRNA), short hairpin RNAs (shRNAs), and other RNA species that can be cleaved in vivo to form siRNAs. RNAi constructs herein also include expression vectors (“RNAi expression vectors”) capable of giving rise to transcripts which form dsRNAs or hairpin RNAs in cells, and/or transcripts which can produce siRNAs in vivo. Further, the iRNA may be asymmetrical iRNA (aiRNA). U.S. Pat. No. 9,328,345. The iRNA agents may be complementary (or antisense) to the coding strand (sense strand) of the mRNA encoding RIOK3, optionally SEQ ID NO: 1. Preferably, the iRNA agent targets a subsection of the mRNA encoding RIOK3, preferably SEQ ID NO: 2.

RNAi expression vectors express (transcribe) RNA which produces siRNA moieties in the cell in which the construct is expressed. Such vectors include a transcriptional unit comprising an assembly of (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA, which can be processed to an siRNA), and (3) appropriate transcription initiation and termination sequences. The choice of promoter and other regulatory elements generally varies according to the intended host cell.

The RNAi constructs contain a nucleotide sequence that hybridizes under physiologic conditions of the cell to the nucleotide sequence of at least a portion of the mRNA transcript for the gene to be inhibited (i.e., a RIOK3-encoding polynucleotide sequence). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 base pairs, or 1 in 10 base pairs, or 1 in 20 base pairs, or 1 in 50 base pairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of a nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis.

The small interfering RNA (siRNA) may be around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group. For example, the siRNA may be about 19, 20, 21, 22, 23, 23, 25, 26, 27, 28, 29, or 30 nucleotides in length. The siRNA may be about 21, 22, or 23 nucleotides in length. For example, the siRNA may target, and optionally cleave SEQ ID NO: 2 (GCTCAGCATTGAGAGAATAAA). The siRNA may bind to SEQ ID NO: 2 (GCTCAGCATTGAGAGAATAAA) leading to the mRNA's degradation.

The RNAi construct may be in the form of a short hairpin structure (shRNA). The shRNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; Yu et al., Proc Natl Acad Sci USA, 2002, 99:6047-52). Often, such shRNAs are engineered in cells or in an animal to ensure continuous and stable suppression of a desired gene. siRNAs can be produced by processing a hairpin RNA in the cell. Moore et al. Methods Mol Biol. (2010) 629:141-158.

A plasmid can be used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA. The iRNAs described herein may decrease RIOK3 by targeting SEQ ID NO: 1, preferably SEQ ID NO: 2.

### Ribozymes

The ribonucleotide agent that decreases expression of RIOK3 may be a ribozyme. Ribozymes molecules designed to catalytically cleave an mRNA transcripts can also be used to prevent translation of mRNA. See, e.g., WO 90/11364; Sarver et al., 1990, Science 247:1222-1225 and U.S. Pat. No. 5,093,246. While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591.

The ribozyme inhibitors described herein may also include RNA endoribonucleases (“Cech-type ribozymes”) such as the one that occurs naturally in Tetrahymena thermophila (known as the IVS, or L-19 IVS RNA) and have been described in the art. Zaug, et al., 1984, Science, 224:574-578; Zaug and Cech, 1986, Science, 231:470-475; Zaug, et al., 1986, Nature, 324:429-433; WO 88/04300; Been and Cech, 1986, Cell, 47:207-216. The Cech-type ribozymes have an 8-basepair active site that hybridizes to a target RNA sequence whereafter cleavage of the target RNA takes place. Cech-type ribozymes that target 8-basepair active site sequences may be used in the methods and compositions described herein. For example, Cech-type ribozymes that target an 8-basepair stretch comprised within SEQ ID NO: 1, preferably SEQ ID NO: 2.

As in the antisense approach, the ribozymes can be composed of modified oligonucleotides (e.g., for improved stability, targeting, serum half-life) and can be delivered to cells in vitro or in vivo. A preferred method of delivery involves using a DNA construct “encoding” the ribozyme under the control of a strong constitutive pol III or pol II promoter, so that transfected cells will produce sufficient quantities of the ribozyme to destroy targeted messages and inhibit translation. Because ribozymes are catalytic, a lower intracellular concentration may be required for efficiency. The ribozymes described herein may decrease RIOK3 by targeting SEQ ID NO: 1, preferably SEQ ID NO: 2. For example, the ribozymes described herein may bind to the RIOK3 mRNA, preferably SEQ ID NO: 2 (GCTCAGCATTGAGAGAATAAA) and cleave the mRNA, leading to degradation of the transcript and a decrease in RIOK3 expression.

microRNA

The ribonucleotide agent that decreases RIOK3 may be a microRNA. MicroRNAs (miRNAs) are a class of non-coding RNAs that play important roles in regulating gene expression. The majority of miRNAs are transcribed from DNA sequences into primary miRNAs and processed into precursor miRNAs, and finally mature miRNAs. In most cases, miRNAs interact with the 3′ untranslated region (3′ UTR) of target mRNAs to induce mRNA degradation and translational repression. However, interaction of miRNAs with other regions, including the 5′ UTR, coding sequence, and gene promoters, have also been reported. Under certain conditions, miRNAs can also activate translation or regulate transcription. The microRNAs described herein may decrease RIOK3 by targeting SEQ ID NO: 1, preferably SEQ ID NO: 2. The microRNA described herein may bind to and, optionally, cleave SEQ ID NO: 2 (GCTCAGCATTGAGAGAATAAA). By binding to and, optionally cleaving, SEQ ID NO: 2 (GCTCAGCATTGAGAGAATAAA) the microRNA causes the mRNA's degradation, reducing RIOK3 expression.

lncRNA

The ribonucleotide agent that decreases RIOK3 may be a long noncoding RNA (lncRNA). LncRNA represent the largest group of non-coding RNAs produced from the genome. LncRNAs are generally described as transcripts >200 nucleotides in length, lacking protein-coding potential. In the most recent GENCODE V30 release, there are 16,193 annotated lncRNAs in the human genome. Robinson et al. Biochim Biophys Acta Gene Regul Mech (2020) 1863 (4): 194419. The lncRNA described herein may decrease RIOK3 by targeting SEQ ID NO: 1, preferably SEQ ID NO: 2.

### Hybridization Conditions

The ribonucleotide agents may hybridize to the RIOK3 mRNA, e.g., the ribonucleic acid sequence of SEQ ID NO: 1, preferably specifically to the ribonucleic acid sequence of SEQ ID NO: 2. For example, antisense ribonucleotide agents may comprise the antisense strand to the ribonucleic acid sequence of SEQ ID NO: 1, preferably specifically to the ribonucleic acid sequence of SEQ ID NO: 2.

Annealing conditions may comprise heating the combination for between about 1-10 minutes, optionally about 5 minutes, at temperature between about 65° C. and 75° C., optionally at about 70° C. After heating, decrease the temperature to between about 40° C. and 50° C., optionally about 45° C., over the course of 1-60 minutes, optionally about 30 minutes. After reaching the second temperature, the mixture is maintained at between about 40° C. and 50° C., optionally about 45° C., for between about 60 and 240 minutes, optionally for about 120 minutes. The mixture may further be agitated for about 1-10 minutes, optionally about 5 minutes. This may be done at a temperature at between about 40° C. and 50° C., optionally about 45° C. The reaction vessel is then incubated at a temperature between about 40° C. and 50° C., optionally about 45° C., for about 1-30 minutes, optionally about 15 minutes. The annealing conditions may further comprise washes, for example, one or more washes in 0.2×SSC/0.1% SDS at about 50-65° C., under highly stringent conditions, e.g., hybridization to filter-bound nucleic acid in 3×SSC at about 45° C. followed by one or more washes in 1×SSC at 20° C. Another wash buffer may comprise 150 mM LiCl, 1% Triton, 1 mM EDTA, 5 mM DTT, 20 mM Tris pH 7.5. Other wash buffers that may be used in annealing reactions included but are not limited to a wash buffer comprising 20 mM Tris-HCl, pH 7.5, 500 mM LiCl, 0.1% LiDS, 1 mM EDTA, 5 mM DTT; a wash buffer comprising 20 mM Tris-HCl, pH 7.5, 500 mM LiCl, 1 mM EDTA; and a low-salt stringent wash buffer comprising 20 mM Tris-HCl, pH 7.5, 200 mM LiCl, 1 mM EDTA; a wash buffer may comprise 0 mM LiCl, 1% Triton, 1 mM EDTA, 5 mM DTT, 20 mM Tris pH 7.5.

Other stringent hybridization conditions which are known in the art may be used. See, for example, Ausubel et al. [Eds.] (1989) Current Protocols in Molecular Biology, Vol. I, Green Publishing Associates, Inc. and John Wiley & Sons, Inc., New York at pages 6.3.1-6.3.6 and 2.10.3.

### Gene Editing

In addition to the ribonucleotide agents described herein, inhibition of RIOK3-mediated cellular signaling by suppression of RIOK3 expression and/or enzymatic activity can be achieved by way of disruption of the genetic sequence encoding the RIOK3 protein. One effective means of targeted gene cleavage is the CRISPR system. For example, hematopoietic stem cells (HSCs) may be removed from a patient, treated using the CRISPR methodology described herein to decrease expression of RIOK3, and then the modified HSCs are returned to the patient. The CRISPR agents may specifically target the ribonucleic acid sequence of SEQ ID NO: 1, preferably specifically to the ribonucleic acid sequence of SEQ ID NO: 2.

The term CRISPR, abbreviation for Clustered Regularly Interspaced Short Palindromic Repeats, was originally coined in reference to segments of prokaryotic DNA that contain short, repetitive base sequences, initially found in bacteria and archaea. In a palindromic repeat, the sequence of nucleotides is the same in both directions. Each repetition is followed by short segments of spacer DNA from previous exposures to foreign DNA (e.g., DNA of a virus). Small clusters of Cas (CRISPR-associated) genes are located next to CRISPR sequences. It was later recognized that the CRISPR/Cas system is a prokaryotic immune system that confers resistance to foreign genetic elements especially those of viral origin and thereby provides a form of acquired immunity. RNA harboring the spacer sequence helps Cas (CRISPR-associated) proteins recognize and cut exogenous DNA. Other RNA-guided Cas proteins cut foreign RNA. CRISPRs are found in approximately 50% of sequenced bacterial genomes and nearly 90% of sequenced archaea, and recently the CRISPR/Cas system have been adapted for use in targeted gene editing in eukaryotic cells. See, e.g., Ledford (2016), Nature 531 (7593): 156-9; U.S. Pat. Nos. 8,697,359; 8,771,945; 8,871,445; and 11,005,799.

A simple version of the CRISPR/Cas system, CRISPR/Cas9, has been modified to edit genomes. By delivering the Cas9 nuclease complexed with a synthetic guide RNA (gRNA) into a cell, typically by transfecting the cell with one or more expression vectors encoding for the Cas9 nuclease and the gRNA, the cell's genome can be cut at a pre-selected location, allowing a target gene (e.g., the RIOK3 gene) to be removed and/or substituted by a new coding sequence.

In the instant case, an expression vector (for example, a viral vector) carrying the coding sequence for a RIOK3-specific gRNA can be introduced into a cell in which the endogenous RIOK3 gene is to be knocked out (for example, an erythroid cell or an erythroid progenitor cell). The same expression vector optionally also carries the coding sequence for the CRISPR/Cas9 nuclease or equivalent. In the alternative, a separate expression vector may be used to introduce the CRISPR/Cas9 nuclease coding sequence for its expression in the target cells. In some cases, more than one (e.g., two) distinct gRNAs are used to ensure removal and/or replacement of a target genomic sequence (e.g., one that encodes the RIOK3 protein). Preferably, the CRISPR/Cas9 system may be used to target the ribonucleotide sequence of SEQ ID NO: 1, more preferably SEQ ID NO: 2.

### RIOK3 Functional Assays

The inhibitors of RIOK3-mediated cellular signaling are useful for their ability to negate the downstream effects of RIOK3 signaling, especially as therapeutics for patients suffering from sickle cell diseases and complications thereof. Assays for confirming such inhibitory effect of an inhibitor can be performed in vitro or in vivo. An in vitro assay typically involves exposure of cultured cells to an inhibitor and monitoring of subsequent biological and biochemical changes in the cells. For example, following exposure to an inhibitor at an adequate concentration for an appropriate amount of time, suitable cells (such as those capable of expressing fetal beta-globin, e.g., erythroid cells or their progenitor cells) are examined for any potential changes in their fetal beta-globin synthesis rate by immunoassays such as Western blot and in situ immunostaining, etc. Further downstream changes due to RIOK3 signaling, e.g., phosphorylation of the MDA5 protein and expression of BCL11A protein, and expression of LRF expression can also be monitored to provide an indication of suppressed signaling via RIOK3. An inhibitory effect is detected when a decrease in RIOK3-mediated signaling, as indicated by any one aforementioned parameter, by at least 10%, 20%, 30%, 40%, 50%, 60%, 70%, 80%, 90% or more is observed.

The enhancing effects on fetal beta-globin synthesis by a RIOK3 inhibiting ribonucleotide agent described herein can also be demonstrated in in vivo assays. For example, a RIOK3 inhibiting ribonucleotide agent described herein can be injected into animals that suffer from a sickle cell disease and therefore show inadequate beta-globin expression and/or activity. Injection methods can be subcutaneous, intramuscular, intravenous, intraperitoneal, or a combination thereof. Changes in disease development is subsequently monitored by various means, such as measuring the level of hemoglobin or number of red blood cells in the circulatory system, in comparison with a control group of animals with similar disease or condition but not given the inhibitor. The Examples section of this disclosure provides detailed description of some exemplary in vivo assays. An inhibitory effect is detected when a positive effect on hemoglobin level or erythrocyte number is established in the test group. Preferably, the positive effect is at least a 10% increase; more preferably, the increase is at least 20%, 30%, 40%, 50%, 60%, 70%, 80%, 90%, 100%, 200%, or higher.

### Compositions

The RIOK3 inhibiting ribonucleotide agent described herein may be formulated into a composition. The compositions comprising a RIOK3 inhibiting ribonucleotide agent described herein may be a pharmaceutical composition. The composition, including pharmaceutical compositions, may comprise an adjuvant, carrier, buffers, antioxidants, wetting agents, lubricating agents, gelling agents, thickening agents, binding agents, disintegrating agents, humectants, preservatives, diluent, stabilizer, filler, excipient, or a combination thereof.

The compositions described herein may be formulated as a pharmaceutical composition comprising a RIOK3 inhibiting ribonucleotide agent and pharmaceutically acceptable carrier. Pharmaceutically acceptable carriers include, but are not limited to, excipient, lubricant, emulsifier, stabilizer, solvent, diluent, buffer, vehicle, or a combination thereof.

Pharmaceutically acceptable carriers are well known in the art and include, for example, aqueous solutions such as water or physiologically buffered saline or other solvents or vehicles such as glycols, glycerol, oils such as olive oil or injectable organic esters. Pharmaceutically acceptable carriers may be a liquid, including but not limited to water and oils, including those of petroleum, animal, vegetable or synthetic origin, such as peanut oil, soybean oil, mineral oil, and sesame oil. The pharmaceutical carriers may be saline, gum acacia, gelatin, starch paste, talc, keratin, colloidal silica, or urea. In addition, auxiliary, stabilizing, thickening, lubricating and coloring agents may be used. For example, sugars and/or amino acids may be admixed into the pharmaceutical composition. The pharmaceutical composition may comprise water, glycerin, phospholipids, or a mixture thereof. Other examples of suitable pharmaceutical carriers are described in Remington's Pharmaceutical Sciences (Alfonso Gennaro ed., Krieger Publishing Company (1997); Remington's: The Science and Practice of Pharmacy, 21^{st} Ed. (Lippincott, Williams & Wilkins (2005); Modern Pharmaceutics, vol. 121 (Gilbert Banker and Christopher Rhodes, CRC Press (2002).

The pharmaceutical carrier can be either solid or liquid. Solid form preparations include, for example, powders, tablets, dispersible granules, capsules, cachets, and suppositories. A solid carrier can be one or more substances that can also act as diluents, flavoring agents, solubilizers, lubricants, suspending agents, binders, or tablet disintegrating agents; it can also be an encapsulating material.

The pharmaceutical compositions can include the formulation of the active compound of a RIOK3 inhibiting ribonucleotide agent with encapsulating material as a carrier providing a capsule in which the inhibitor (with or without other carriers) is surrounded by the carrier, such that the carrier is thus in association with the compound. In a similar manner, cachets can also be included. Tablets, powders, cachets, and capsules can be used as solid dosage forms suitable for oral administration.

Liquid pharmaceutical compositions include, for example, solutions suitable for oral or parenteral administration, suspensions, and emulsions suitable for oral administration. Sterile water solutions of the active component, e.g., a RIOK3 inhibiting ribonucleotide agent, or sterile solutions of the active component in solvents comprising water, buffered water, saline, PBS, ethanol, or propylene glycol are examples of liquid compositions suitable for parenteral administration. The compositions may contain pharmaceutically acceptable auxiliary substances as required to approximate physiological conditions, such as pH adjusting and buffering agents, tonicity adjusting agents, wetting agents, detergents, and the like.

Single or multiple administrations of the compositions can be carried out with dose levels and pattern being selected by the treating physician. In any event, the pharmaceutical formulations should provide a quantity of a RIOK3 inhibiting ribonucleotide agent sufficient to effectively inhibit cellular signaling mediated by RIOK3 in the patient, either therapeutically or prophylactically.

### Microparticle/Nanoparticle Delivery

A RIOK3 inhibiting ribonucleotide agent described herein can be encapsulated within a microparticle and/or nanoparticle, dispersed within the polymer matrix that forms the microparticle and/or nanoparticle, covalently or non-covalently associated with the surface of the microparticle and/or nanoparticle or combinations thereof.

The term “microspheres” is art recognized and includes substantially spherical colloidal structures, e.g., formed from biocompatible polymers such as subject compositions, having a size ranging from about one or greater up to about 1,000 microns (μm). In general, “microcapsules,” also an art recognized term, are distinguished from microspheres, because microcapsules are generally covered by a substance of some type, such as a polymeric formulation. The term “microparticles” is also art recognized, and includes microspheres and microcapsules, as well as structures that may not be readily placed into either of the above two categories, all with dimensions on average of less than about 1,000 microns. A microparticle may be spherical or non-spherical and may have any regular or irregular shape. If the structures are less than about one micron in diameter, then the corresponding art recognized terms “nanosphere,” “nanocapsule” and “nanoparticle” (which includes nanospheres and nanocapsules) may be utilized. In certain embodiments, the nanospheres, nanocapsules and nanoparticles have an average diameter of about 500 nm, 200 nm, 100 nm, 50 nm, 10 nm or 1 nm, e.g., when measured by dynamic light scattering (DLS), transmission electron microscopy, scanning electron microscopy, or another method. In some embodiments, the average diameter of the particles is from about 200 nm to about 600 nm, e.g., from about 200 nm to about 500 nm.

A composition comprising microparticles or nanoparticles can include particles of a range of particle sizes. In certain embodiments, the particle size distribution may be uniform, e.g., within less than about a 20% standard deviation of the mean volume diameter, and in other embodiments, still more uniform, e.g., within about 10%, 8%, 5%, 3% or 2% of the median volume diameter.

The microparticles and/or nanoparticles (the particles) can be used for in vivo and/or in vitro delivery of a RIOK3 inhibiting ribonucleotide agent described herein.

The particles may also include antigens and/or adjuvants (e.g., molecules enhancing an immune response, such as Glucopyranosyl Lipid Adjuvant (GLA), CpG oligodeoxynucleotides (e.g., Class A or B), poly (I:C), aluminum hydroxide, and Pam3CSK4).

In some embodiments, particles comprising a RIOK3 inhibiting ribonucleotide agent may contain less than 80%, less than 75%, less than 70%, less than 60%, less than 50% by weight, less than 40% by weight, less than 30% by weight, less than 20% by weight, less than 15% by weight, less than 10% by weight, less than 5% by weight, less than 1% by weight, less than 0.5% by weight, or less than 0.1% by weight of the RIOK3 inhibiting ribonucleotide agent, but greater than 0%.

The particles comprising a RIOK3 inhibiting ribonucleotide agent may contain more than 0.1%, more than 0.5%, more than 1%, more than 5%, more than 10%, more than 15%, more than 20%, more than 30%, more than 40%, more than 50%, more than 60%, more than 70%, more than 75%, or more than 80% by weight of the a RIOK3 inhibiting ribonucleotide agent, but less than 100%.

The composition comprising a RIOK3 inhibiting ribonucleotide agent may further comprise at least one pharmaceutically active agent. The percent loading is dependent on a variety of factors, including the pharmaceutically active agent to be encapsulated, the polymer used to prepare the particles, and/or the method used to prepare the particles.

For example, the delivery method may comprise nanoparticles comprising the ribonucleotide agent described herein and a cationic polymer, lipid nanoparticles comprising the ribonucleotide agent described herein and a cationic/ionizable lipid, and other hydrophobic moieties (e.g., cholesterol). Additionally, base, sugar, and linker modifications may be used to prepare the ribonucleotide agents described herein for delivery to a patient. Kaczmarek et al. Genome Medicine (2017) 9:60; Roberts et al. Nature Reviews (2020) 19:673-694.

### Routes of Administration

The compositions comprising a RIOK3 inhibiting ribonucleotide agent described herein may be administered subcutaneously, intramuscularly, intravenously, intraperitoneally, intrapleurally, intravesicularly, intrathecally, topically, orally, rectally, vaginally, nasally, or by a route as necessitated condition. The compositions comprising a RIOK3 inhibiting ribonucleotide agent described herein may be infused into a subject. The composition comprising a RIOK3 inhibiting ribonucleotide agent described herein may be administered by parenteral administration. One route of administration is intravenous.

### Nucleic Acids, Vectors, and Host Cells

The present invention also provides for an isolated nucleic acid molecule encoding a RIOK3 inhibiting ribonucleotide agent described herein. Nucleic acid molecules that encode the RIOK3 inhibiting ribonucleotide agents described herein. The nucleic acids may be present in whole cells, in a cell lysate, or in a partially purified or substantially pure form. A nucleic acid may be isolated by purification away from other cellular components or other contaminants (e.g. other cellular nucleic acids or proteins) by standard techniques, including alkaline/SDS treatment, CsCI banding, column chromatography, agarose gel electrophoresis and others well known in the art. See Ausubel, et al. (2011) Current Protocols in Molecular Biology John Wiley & Sons, Inc. A nucleic acid described herein may be, for example, DNA or RNA and may or may not contain intronic sequences. The nucleic acid may be a cDNA molecule. Nucleic acids described herein may be obtained using standard molecular biology techniques. Specifically, degenerate codon substitutions may be achieved by generating, e.g., sequences in which the third position of one or more selected codons is substituted with mixed-base and/or deoxyinosine residues. Batzer, et al. (1991) Nucleic Acid Res. 19:5081; Ohtsuka, et al. (1985) J. Biol. Chem. 260:2605-08; Rossolini, et al. (1994) Mol. Cell. Probes 8:91-98.

### Treatment of Sickle Cell Diseases and Complications Thereof

The RIOK3 inhibiting ribonucleotide agent described herein may be used for the treatment of sickle cell disease (SCD). SCD is a group of inherited red blood cell disorders, including but not limited to hemoglobin SS disease, hemoglobin SC disease, hemoglobin SB+ (beta) thalassemia, hemoglobin SB 0 (beta-zero) thalassemia, hemoglobin SD, hemoglobin SE, and hemoglobin SO.

The most common sickle cell diseases are Sickle Cell Anemia (SS), Sickle Hemoglobin-C Disease (SC), Sickle Beta-Plus Thalassemia and Sickle Beta-Zero Thalassemia.

Sickle Cell Anemia (SS): When a child inherits one substitution beta globin genes (the sickle cell gene) from each parent, the child has Sickle Cell Anemia (SS). Populations that have a high frequency of sickle cell anemia are those of African and Indian descents.

Sickle Hemoglobin-C Disease (SC): Individuals with Sickle Hemoglobin-C Disease (SC) inherit two abnormal copies of the beta-globin gene; one copy of the sickle mutation and one copy of the C mutation. Sickle Hemoglobin-C disease may cause similar symptoms as sickle cell anemia but less anemia due to a higher blood count level. Populations that have a high frequency of Sickle Hemoglobin-C disease are those of West African, Mediterranean and Middle Eastern descents.

Sickle Beta-Plus Thalassemia: Individuals with Sickle Beta Thalassemia (SB) disease also contain substitutions in both beta globin genes. The severity of the disease varies according to the amount of normal beta globin produced. When no beta globin is produced, the symptoms are almost identical to sickle cell anemia, with severe cases needing chronic blood transfusions. Populations that have a high frequency of Sickle Beta Thalassemia are those of Mediterranean and Caribbean descents.

Sickle Hemoglobin-D Disease: Through research, hemoglobin D, which is a different substitution of the beta globin gene, has been found to interact with the sickle hemoglobin gene. Individuals with Sickle Hemoglobin-D disease (SD) have moderately severe anemia and occasional pain episodes. Populations that have a high frequency of Sickle Hemoglobin-D disease are those of Asian and Latin American descents.

Sickle Hemoglobin-O Disease: Hemoglobin O, another type of substitution in the beta globin gene, also interacts with sickle hemoglobin. Individuals with Sickle Hemoglobin-O disease (SO) can have symptoms of sickle cell anemia. Populations that have a high frequency of Sickle Hemoglobin-O disease are those of Arabian, North African and Eastern Mediterranean descents.

The inventors surprisingly discovered that the RIOK3 expression inhibiting ribonucleotide agent described herein may be used for the treatment of sickle cell disease (SCD). Without wishing to be bound to a theory, the RIOK3 inhibiting ribonucleotide agent described herein decrease the expression of RIOK3, leading to a decrease in beta-hemoglobin (HBB) expression and an increase in gamma-hemoglobin (HBG1, HBG2) expression.

### Sickle Cell Disease Complications—Sickle Cell Crisis

The RIOK3 inhibiting ribonucleotide agent described herein may be used for the prevention of the complications of sickle cell disease (SCD). If there is an increased level of fetal hemoglobin, the complications of SCD are reduced including but not limited to vaso-occlusive crisis, acute chest syndrome, aplastic crisis, hemolytic crisis, dactylitis, seizure, stroke, ischemia, transient ischemic attack, ischemic colitis, or a combination thereof.

The terms “sickle cell crisis” or “sickling crisis” may be used to describe several independent acute conditions occurring in patients with sickle cell disease (SCD). SCD results in anemia and crises that could be of many types including the vaso-occlusive crisis, aplastic crisis, sequestration crisis, hemolytic crisis, and others. Most episodes of sickle cell crises last between five and seven days and includes the need for pain medications and often hospitalization. Although infection, dehydration, and acidosis (all of which favor sickling) can act as triggers, in most instances, no predisposing cause is identified. Kumar et al. (2009) Robbins and Cotran Pathologic Basis of Disease, Professional Edition: Expert Consult—Online (Robbins Pathology) Elsevier Health. Kindle Edition.

The RIOK3 expression inhibiting ribonucleotide agent described herein may be used for the treatment of sickle cell disease (SCD), preferably a complication of sickle cell anemia, including but not limited to sickle cell crisis, vaso-occlusive crisis, aplastic crisis, hemolytic crisis, dactylitis, acute chest syndrome, seizure, stroke, ischemia, transient ischemic attack, ischemic colitis, or a combination thereof.

### Treatment of Malaria

Infants expressing gamma-hemoglobin (also referred to as hemoglobin F) show a resistance to the development of malaria, caused by infection by Plasmodium falciparum. Pasvol et al. Lancet (1976) 1 (7972): 1269-72. Although the mechanism is unclear, an increase in the expression of gamma hemoglobin (hemoglobin F) may be protective against malarial infection. Accordingly, the RIOK3 inhibiting ribonucleotide agent described herein may be used in methods for treating malaria, including preventing malaria.

### Vectors for Gene Delivery

For delivery to a cell or organism, a polynucleotide encoding an RIOK3 inhibiting ribonucleotide agent described herein can be incorporated into a vector. Examples of vectors used for such purposes include expression plasmids capable of directing the expression of the nucleic acids in the target cell. The vector may be a viral vector system wherein the polynucleotide is incorporated into a viral genome that is capable of transfecting the target cell. In one embodiment, the encoding polynucleotide can be operably linked to expression and control sequences that can direct expression of the polypeptide or oligonucleotide in the desired target host cells. Thus, one can achieve expression of the polypeptide or oligonucleotide inhibitor under appropriate conditions in the target cell.

### Gene Delivery Systems

Viral vector systems useful in the expression of a RIOK3 inhibiting ribonucleotide agent described herein include, but are not limited to, naturally occurring or recombinant viral vector systems. Depending upon the particular application, suitable viral vectors include replication competent, replication deficient, and conditionally replicating viral vectors. For example, viral vectors can be derived from the genome of human or bovine adenoviruses, vaccinia virus, herpes virus, adeno-associated virus, minute virus of mice (MVM), HIV, sindbis virus, and retroviruses (including but not limited to Rous sarcoma virus and lentivirus), and MoMLV. Typically, the coding sequence of interest (e.g., one encoding for a RIOK3 inhibiting ribonucleotide agent described herein) are inserted into such vectors to allow packaging of the gene construct, typically with accompanying viral DNA, followed by infection of a sensitive host cell and expression of the coding sequence of interest.

A gene delivery system may be any means for the delivery of a polynucleotide sequence encoding a RIOK3 inhibiting ribonucleotide agent described herein to a target cell. The nucleic acids may be conjugated to a cell receptor ligand for facilitated uptake (e.g., invagination of coated pits and internalization of the endosome) through an appropriate linking moiety, such as a DNA linking moiety (Wu et al., J. Biol. Chem. 263:14621-14624 (1988); WO 92/06180), or by ultrasound-microbubble delivery system (Lan H Y et al., J. Am Soc. Nephrol. 14:1535-1548). For example, nucleic acids can be linked through a polylysine moiety to asialoorosomucoid, which is a ligand for the asialoglycoprotein receptor of hepatocytes.

Similarly, viral envelopes used for packaging gene constructs that include the nucleic acids of the invention can be modified by the addition of receptor ligands or antibodies specific for a receptor to permit receptor-mediated endocytosis into specific cells. See, e.g., WO 93/20221, WO 93/14188, and WO 94/06923. The DNA constructs encoding a RIOK3 inhibiting ribonucleotide agent described herein may be linked to viral proteins, such as adenovirus particles, to facilitate endocytosis (Curiel et al., Proc. Natl. Acad. Sci. U.S.A. 88:8850-8854 (1991)), microtubule inhibitors (WO/9406922), synthetic peptides mimicking influenza virus hemagglutinin (Plank et al., J. Biol. Chem. 269:12918-12924 (1994)), and nuclear localization signals such as SV40 T antigen (WO93/19768).

Retroviral vectors may also be useful for introducing the coding sequence of a RIOK3 inhibiting ribonucleotide agent described herein into target cells or patients. Retroviral vectors are produced by genetically manipulating retroviruses. The viral genome of retroviruses is RNA. Upon infection, this genomic RNA is reverse transcribed into a DNA copy which is integrated into the chromosomal DNA of transduced cells with a high degree of stability and efficiency. The integrated DNA copy is referred to as a provirus and is inherited by daughter cells as is any other gene. The wild type retroviral genome and the proviral DNA have three genes: the gag, the pol and the env genes, which are flanked by two long terminal repeat (LTR) sequences. The gag gene encodes the internal structural (nucleocapsid) proteins; the pol gene encodes the RNA directed DNA polymerase (reverse transcriptase); and the env gene encodes viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site). See, Mulligan, In: Experimental Manipulation of Gene Expression, Inouye (ed), 155-173 (1983); Mann et al., Cell 33:153-159 (1983); Cone and Mulligan, Proceedings of the National Academy of Sciences, U.S.A., 81:6349-6353 (1984)).

The design of retroviral vectors is known in the art. In brief, if the sequences necessary for encapsidation (or packaging of retroviral RNA into infectious virions) are missing from the viral genome, the result is a cis acting defect which prevents encapsidation of genomic RNA. However, the resulting mutant is still capable of directing the synthesis of all virion proteins. Retroviral genomes from which these sequences have been deleted, as well as cell lines containing the mutant genome stably integrated into the chromosome are well known in the art and are used to construct retroviral vectors. Preparation of retroviral vectors and their uses are described in the art. European Patent Application 0 178 220; U.S. Pat. No. 4,405,712; Gilboa Biotechniques 4:504-512 (1986); Mann et al., Cell 33:153-159 (1983); Cone and Mulligan Proc. Natl. Acad. Sci. USA 81:6349-6353 (1984); Eglitis et al. Biotechniques 6:608-614 (1988); Miller et al. Biotechniques 7:981-990 (1989); Miller (1992) supra; Mulligan (1993), supra; and WO 92/07943.

The retroviral vector particles are prepared by recombinantly inserting the desired nucleotide sequence into a retrovirus vector and packaging the vector with retroviral capsid proteins by use of a packaging cell line. The resultant retroviral vector particle is incapable of replication in the host cell but is capable of integrating into the host cell genome as a proviral sequence containing the desired nucleotide sequence. As a result, the patient is capable of producing, for example, a polypeptide or polynucleotide inhibitor of the invention and thus restore the target cells (e.g., erythroid cells) to a normal phenotype.

Packaging cell lines that are used to prepare the retroviral vector particles are typically recombinant mammalian tissue culture cell lines that produce the necessary viral structural proteins required for packaging, but which are incapable of producing infectious virions. The defective retroviral vectors that are used, on the other hand, lack these structural genes but encode the remaining proteins necessary for packaging. To prepare a packaging cell line, one can construct an infectious clone of a desired retrovirus in which the packaging site has been deleted. Cells comprising this construct will express all structural viral proteins, but the introduced DNA will be incapable of being packaged. Alternatively, packaging cell lines can be produced by transforming a cell line with one or more expression plasmids encoding the appropriate core and envelope proteins. In these cells, the gag, pol, and env genes can be derived from the same or different retroviruses.

A number of packaging cell lines suitable for the present invention are also available in the prior art. Examples of these cell lines include Crip, GPE86, PA317 and PG13 (see Miller et al., J. Virol. 65:2220-2224 (1991)). Examples of other packaging cell lines are described in Cone and Mulligan Proceedings of the National Academy of Sciences, USA, 81:6349-6353 (1984); Danos and Mulligan Proceedings of the National Academy of Sciences, USA, 85:6460-6464 (1988); Eglitis et al. (1988), supra; and Miller (1990), supra.

Packaging cell lines capable of producing retroviral vector particles with chimeric envelope proteins may be used. Alternatively, amphotropic or xenotropic envelope proteins, such as those produced by PA317 and GPX packaging cell lines may be used to package the retroviral vectors.

### Administration of Compositions

Compositions comprising RIOK3 inhibiting ribonucleotide agent described herein or a polynucleotide sequence encoding a RIOK3 inhibiting ribonucleotide agent described herein can be delivered to target tissue or organ using any delivery method known in the art. The compositions described herein may be formulated for subcutaneous, intramuscular, intravenous, or intraperitoneal injection, or for oral ingestion or for topical application.

The compositions described herein may be administered to a cell. The cell can be provided as part of a tissue, such as erythrocytes as a part of the circulatory system, or as an isolated cell, such as in tissue culture. The cell can be provided in vivo, ex vivo, or in vitro. For example, hematopoietic stem cells (HSCs) may be removed from a patient, treated with a RIOK3 inhibiting ribonucleotide agent described herein to change the hemoglobin profile, and then, the modified HSCs may be reintroduced into the patient. The HSCs may be allogeneic or autologous.

The compositions described herein can be introduced into the tissue of interest in vivo or ex vivo by a variety of methods. Nucleic acids encoding a RIOK3 inhibiting ribonucleotide agent described herein, or a RIOK3 inhibiting ribonucleotide agent described herein, may be introduced into cells by such methods as microinjection, calcium phosphate precipitation, liposome fusion, ultrasound, electroporation, biolistics, or a combination thereof. The nucleic acids encoding a RIOK3 inhibiting ribonucleotide agent described herein, or RIOK3 inhibiting ribonucleotide agent described herein, may be taken up directly by the tissue of interest, for example, when the targeted cells are the red blood cells intravenous injection is appropriate.

The RIOK3 inhibiting ribonucleotide agent described herein may be administered ex vivo to cells or tissues explanted from a patient, then returned to the patient. Examples of ex vivo administration of therapeutic gene constructs include Nolta et al., Proc Natl. Acad. Sci. USA 93 (6): 2414-9 (1996); Koc et al., Seminars in Oncology 23 (1): 46-65 (1996); Raper et al., Annals of Surgery 223 (2): 116-26 (1996); Dalesandro et al., J. Thorac. Cardi. Surg., 11 (2): 416-22 (1996); and Makarov et al., Proc. Natl. Acad. Sci. USA 93 (1): 402-6 (1996).

Effective dosage of the formulations will vary depending on many different factors, including means of administration, target site, physiological state of the patient, and other medicines administered. Thus, treatment dosages will need to be titrated to optimize safety and efficacy. In determining the effective amount of the vector to be administered, the physician should evaluate the particular nucleic acid used, the disease state being diagnosed; the age, weight, and overall condition of the patient, circulating plasma levels, vector toxicities, progression of the disease, and the production of anti-vector antibodies. The size of the dose also will be determined by the existence, nature, and extent of any adverse side-effects that accompany the administration of a particular vector.

Doses may generally range between about 0.01 and about 100 μg per kilogram of body weight, preferably between about 0.1 and about 50 μg/kg of body weight or about 10⁸-10¹⁰ or 10¹² particles per injection. In general, the dose equivalent of a naked nucleic acid from a vector is from about 1 μg-100 μg for a typical 70 kg patient, and doses of vectors which include a retroviral particle are calculated to yield an equivalent amount of nucleic acid encoding a RIOK3 inhibiting ribonucleotide agent described herein.

### Kits

The invention also provides kits for inhibiting RIOK3 signaling and treating sickle cell diseases are also described. The kits typically include a container that contains (1) a pharmaceutical composition having an effective amount of an RIOK3 inhibiting ribonucleotide agent described herein and (2) informational material containing instructions on how to dispense the pharmaceutical composition, including description of the type of patients who may be treated (e.g., human patients suffering from sickle cell disease or beta-thalassemia), the schedule (e.g., dose and frequency) and route of administration, and the like. In some cases, a second container is included in the kit to provide a second pharmaceutical composition comprising an effective amount of a second inhibitor of RIOK3.

### EXAMPLES

### Example 1

### Knockdown of RIOK3 RNA Increases Gamma-Hemoglobin Expression and Decreases Beta-Hemoglobin Expression

Quantitative PCR shows reduced RIOK3 RNA level after shRNA knockdown. CD34+ hematopoietic stem and progenitor cell derived erythroblasts were transduced on day 2 of culture with a control shRNA lentiviral vector (shNC) or a RIOK3 specific lentiviral vector (shRIOK3; SEQ ID NOS: 3 and 4) and RIOK3 expression analyzed on day 12 of culture by Q-PCR. The data shows that the RIOK3 shRNA efficiently targets and reduces RIOK3 RNA.

RIOK3 knockdown leads to upregulation of fetal beta-globin expression. CD34+ hematopoietic stem and progenitor cell derived erythroblasts were transduced on day 2 of culture with a control shRNA lentiviral vector (shNC) or a RIOK3 specific lentiviral vector (shRIOK3) and globin (HBB, HBA, HBG1 and HBG2) levels analyzed on day 11 of culture by high performance liquid chromatography (HPLC). % HbF was calculated by dividing HBG1+HBG2 values with total β-globin (HBB+HBG1+HBG2) values. The data shows that upon RIOK3 knockdown the levels of fetal β-globins HBG1 and HBG2 are robustly increased compared to control.

RIOK3 knockdown leads to downregulation of BCL11A and LRF expression. CD34+ hematopoietic stem and progenitor cell derived erythroblasts were transduced on day 2 of culture with a control shRNA lentiviral vector (shNC) or a RIOK3 specific lentiviral vector (shRIOK3) and BCL11A and LRF expression analyzed on day 12 of culture by Q-PCR. The data shows that upon RIOK3 knockdown the fetal hemoglobin repressors BCL11A and LRF are significantly decreased at the transcriptional level.

RIOK3 knockdown leads to downregulation of BCL11A and LRF protein expression in erythroid progenitor cells. CD34+ hematopoietic stem and progenitor cell derived erythroblasts were transduced on day 2 of culture with a control shRNA lentiviral vector (shNC) or a RIOK3 specific lentiviral vector (shRIOK3) and BCL11A and LRF protein levels analyzed on day 12 of culture by Western blotting. The data shows that BCL11A and LRF protein levels are significantly reduced upon RIOK3 knockdown compared to control.

RIOK3 knockdown in primary CD34+ derived erythroid cells. Cytospin shows no morphological differences on day 15 of culture between cells transduced with control shRNA vs cells transduced with RIOK3 specific shRNA. CD34+ hematopoietic stem and progenitor cell derived erythroblasts were transduced on day 2 of culture with a control shRNA lentiviral vector (shNC) or a RIOK3 specific lentiviral vector (shRIOK3) and spun on glass slides and stained with the HEMA 3 manual staining system.

Taken together, the data demonstrates that a RIOK3 inhibiting ribonucleotide agent described herein may be used to decrease expression of RIOK3, and, subsequently, beta-hemoglobin (HBB) and increase the expression of gamma-hemoglobin.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

1. A ribonucleotide agent that decreases expression of serine/threonine-protein kinase 3 (RIOK3), wherein the ribonucleotide agent comprises:
an antisense oligonucleotide, short hairpin RNA (shRNA), small interfering RNA (siRNA), an asymmetrical iRNA (aiRNA), a microRNA, a miniRNA, a lncRNA, or a ribozyme, or any combination thereof; and
a forward sequence comprising the nucleotide sequence of SEQ ID NO: 3, or a reverse sequence comprising the nucleotide sequence of SEQ ID NO: 4, or both,
wherein the ribonucleotide agent targets the mRNA sequence of SEQ ID NO: 1, or SEQ ID NO: 2, or both.

2. (canceled)

3. The ribonucleotide agent of claim 1, wherein the ribonucleotide agent is a short hairpin RNA (shRNA); and comprises the forward sequence comprising the nucleotide sequence of SEQ ID NO: 3, and the reverse sequence comprising the nucleotide sequence of SEQ ID NO: 4.

4-11. (canceled)

12. The ribonucleotide agent of claim 1, wherein the ribonucleotide agent targets the mRNA sequence of SEQ ID NO: 2.

13. The ribonucleotide agent of claim 12, wherein the ribonucleotide agent further targets flanking sequences 1-20 ribonucleotides 3′ and/or 5′ of SEQ ID NO: 2.

14. A composition comprising the ribonucleotide agent of claim 1.

15-16. (canceled)

17. A microparticle comprising the ribonucleotide agent of claim 1.

18-19. (canceled)

20. A composition comprising the microparticle of claim 17.

21. (canceled)

22. A vector comprising a polynucleotide encoding the ribonucleotide agent of claim 1.

23-24. (canceled)

25. The vector of claim 22, wherein the viral vector is a lentiviral vector.

26-31. (canceled)

32. The vector of claim 22, wherein the vector is an expression vector.

33. A host cell comprising the vector of claim 32.

34. A method for treating a sickle cell disease in a patient comprising administration of an effective amount of the ribonucleotide agent of claim 1.

35. The method of claim 34, wherein the sickle cell disease is hemoglobin SS disease, hemoglobin SC disease, hemoglobin SB+ (beta) thalassemia, hemoglobin SB 0 (beta-zero) thalassemia, hemoglobin SD, hemoglobin SE, or hemoglobin SO.

36. The method of claim 34, wherein the sickle cell disease is Sickle Cell Anemia (SS), Sickle Hemoglobin-C Disease (SC), Sickle Beta-Plus Thalassemia or Sickle Beta-Zero Thalassemia.

37. A method for treating a complication of sickle cell disease in a patient comprising administration of an effective amount of the ribonucleotide agent of claim 1.

38. The method of claim 37, wherein the complication of sickle cell disease is sickle cell crisis, vaso-occlusive crisis, acute chest syndrome, aplastic crisis, hemolytic crisis, dactylitis, acute chest syndrome, seizure, stroke, ischemia, transient ischemic attack, ischemic colitis, or a combination thereof.

39. A method for promoting fetal beta-globin synthesis in a cell comprising administration of an effective amount of the ribonucleotide agent of claim 1.

40. An ex vivo method for treating a sickle cell disease in a patient in need thereof comprising
(a) obtaining hematopoietic stem and progenitor cells from a patient with a sickle cell disease;
(b) administration of an effective amount of the ribonucleotide agent of claim 1 to the hematopoietic stem and progenitor cells to transfect the cells with the ribonucleotide agent; and
(c) returning the transfected hematopoietic stem and progenitor cells to the patient.

41-51. (canceled)

52. An isolated polynucleotide comprising the nucleic acid sequence of SEQ ID NO: 3, or the nucleic acid sequence of SEQ ID NO: 4, or both.

53-55. (canceled)

56. The ribonucleotide agent of claim 3, wherein the short hairpin RNA (shRNA) is cleavable in vivo to form a small interfering RNA (siRNA).


SEQUENCE LISTING (5 sequences)

SEQ ID NO: 1 - DNA, 3574 bp, Homo sapiens
agttccggacgcggccgccgccgtcgccgccatctgtcacctccactccggcatcagcagccagtcgcccgtgtcccgcctgtctcctcggcggagcctgctgcccgtcctgccacctctctgctctgttcttgtctctgccttcattcccgaatggatctggtaggagtggcatcgcctgagcccgggacggcagcggcctggggacccagcaagtgtccatgggctattcctcaaaatacaatatcttgttctttggctgatgtaatgagtgaacagctggccaaagaattgcagttagaagaagaagctgccgtttttcctgaagttgctgttgctgaaggaccatttattactggagaaaacattgatacttccagtgaccttatgctggctcagatgctacagatggaatatgacagagaatatgatgcacagcttaggcgtgaagaaaaaaaattcaatggagatagcaaagtttccatttcctttgaaaattatcgaaaagtgcatccttatgaagacagcgatagctctgaagatgaggttgactggcaggatactcgtgatgatccctacagaccagcaaaaccggttcccactcctaaaaagggctttattggaaaaggaaaagatatcaccaccaaacatgatgaagtagtatgtgggagaaagaacacagcaagaatggaaaattttgcacctgagtttcaggtaggagatggaattggaatggatttaaaactatcaaaccatgttttcaatgctttaaaacaacatgcctactcagaagaacgtcgaagtgcccgcctacatgagaaaaaggagcattctacagcagaaaaagcagttgatcctaagacacgtttacttatgtataaaatggtcaactctggaatgttggagacaatcactggctgtattagtacaggaaaggagtctgttgtctttcatgcatatggagggagcatggaggatgaaaaggaagatagtaaagttatacctacagaatgtgccatcaaggtatttaaaacaacccttaatgaatttaagaatcgtgacaaatatattaaagatgatttcaggtttaaagatcgcttcagtaaactaaatccacgtaagatcatccgcatgtgggcagaaaaagaaatgcacaatctcgcaagaatgcagagagctggaattccttgtccaacagttgtactactgaagaaacacattttagttatgtcttttattggccatgatcaagttccagcccctaaattaaaagaagtaaagctcaatagtgaagaaatgaaagaagcctactatcaaactcttcatttgatgcggcagttatatcatgaatgtacgcttgtccatgctgacctcagtgagtataacatgctgtggcatgctggaaaggtctggttgatcgatgtcagtcagtcagtagaacctacccaccctcacggcctggagttcttgttccgggactgcaggaatgtctcgcagtttttccagaaaggaggagtcaaggaagcccttagtgaacgagaactcttcaatgctgtttcaggcttaaacatcacagcagataatgaagctgattttttagctgagatagaagctttggagaaaatgaatgaagatcacgttcagaagaatggaaggaaagctgcttcatttttgaaagatgatggagacccaccactactatatgatgaatagcactaatacccactgcttcagtgttaacacagcagtgattgtcagctgccaatagcaaatgaagttatgggtgacttgaaataccaaaacctgaggagtgggcaatggtgcttctgtgcttttcccccttgtaacccatgtgccagatgtgtggaatttttagctcagcattgagagaataaaatgtcactacctctcatcttatgaacaggataatataattctttaacagctataggttatctggctgaagtagacctaattttatgtgacttgtggtgtaaaatgtcttgatgataatttttaaaacttgggtaacacttccaaatatgggaggaaaggacagatgtgtttacaagggaggattttacaacatacttgctttattcacctccctgttttgtgttgcgtctttccttgaatattttattggcccagagttagcctttctcaattatgtttccagactgtggccgtgattctaaaggaaaatgtgtgctctttagtgggtagaacaaatggaaatttggtttcagaatggctgacagaaatcgacataagtcatgtaatttttgttgatatatcatgaaaatgaacagaattctttttccatacttatatctaagaaaaggcatcataggtttctgaaagagataactatataacagctttttaactatccagtcaactttcagcttttctacatttaggtaaaatggttaggatataactcatggtgtggctaatctacatttatcaataaaatgtaaattatctgaaaggacagaatataagatttaaccatgtttgacgtattttaatttagttaatgaagcaaaattcagtttatatttcactagaactgtgtacttgattgattttcagagaaatatcacaaattagaaatattaaatctaaggatgaaaggtatatataaaacaatttgggggccaggcacgatggctcaaacctgtaatcccagcactttgggagaccaaggcgggtggatcacttgaggtcaggagttcaagaccagcctgggcaacatggcgaaaccctgtctctactaaaaatacaaaaattagccgggtgtggtggcacttctctgtaatctcagcttctcaggaggctgagacaggagaatcgcttgaacccgggaggcagaggttgcagtgagctgagatcatgccactgcactccggcctaggtgacagagggaaactccatctccaggaaaaaaaaaaaaaaacccaatttggataccaaattaatcaactaatttgagctatctggccttactcttagtagtttttagtacgtgctggacaccacttttaaaaagcaatcactgtgctagaaaagtatattggctttgttaggattaaagttcattaacttcaatgtaatcatgcctcctattactgaagtcagattggaaccactaaagatccaaactttctgtctggtaatagaaagtaaaaatctagacatcatttacatttgagaaagctgtttttaacattattttaaaatgccaaatatgttctttctagaaaaatatttatttttgtttttgttggatagcttttaattacatttcagagaggtgtaattttgggtagatgctcattacatttttgaaaggtttatgattccaaaataaagatttatatgactggtgatactggctttacagaaatttcagagaactaatttttaaaatctttagcatttaaaactttttttgttttgttttctgacatattctgacaaagagcagcaaaccactgctgtgtggcattcttggagtgtgctgtgaatgtgctttttaagaaattaaaaagagatcattttaaaattg

SEQ ID NO: 2 - DNA, 21 bp, Artificial Sequence
  synthetic oligonucleotide
gctcagcattgagagaataaa

SEQ ID NO: 3 - DNA, 58 bp, Artificial Sequence
  shRNA forward sequence
ccgggctcagcattgagagaataaactcgagtttattctctcaatgctgagctttttg

SEQ ID NO: 4 - DNA, 58 bp, Artificial Sequence
  shRNA reverse sequence
aattcaaaaagctcagcattgagagaataaactcgagtttattctctcaatgctgagc

SEQ ID NO: 5 - DNA, 21 bp, Artificial Sequence
  synthetic oligonucleotide reverse sequence
tttattctctcaatgctgagc